(19) United States
(12) Patent Application Publication
(10) Pub. No.: US 2002/0035758 A1
(43) Pub. Date: Mar. 28, 2002

(54) Hair colouring compositions and their use

(76) Inventor: Dominic Pratt, Bracknell (GB)

Correspondence Address: THE PROCTER & GAMBLE COMPANY, PATENT DIVISION, SHARON WOODS TECHINICAL CENTER, 11511 REED HARTMAN HIGHWAY, CINCINNATI, OH, 45241, US

(21) Appl. No.: 09/974,254

(22) Filed: Oct. 10, 2001

Related U.S. Application Data
(63) Continuation of application No. 09/424,477, filed on Nov. 23, 1999.

(30) Foreign Application Priority Data
May 23, 1997 (GB) 9710756.9
May 23, 1997 (GB) 9710754.4
May 23, 1997 (GB) 9710757.7
May 23, 1997 (GB) 9710759.3
May 23, 1997 (GB) 9710761.9

(51) Int. Cl. A61K 7/13

(52) U.S. Cl. 8/405; 8/406; 8/408; 8/421

(57) ABSTRACT

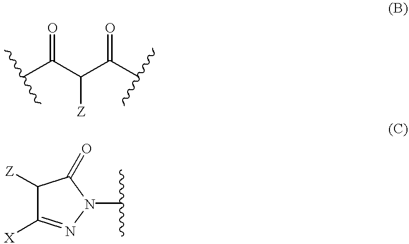

Hair coloring compositions which are of reduced harshness to skinand hair are provided which comprise: (i) one or more developers selected from aminoaromatic systems capable of being oxidized and thereafter undergoing a single electrophilic attack, and (ii) one or more couplers selected from (A) phenols and naphthols having an active leaving group in the para position relative to the hydroxy group, (B) 1,3-diketone derivatives having an active leaving group, or (C) pyrazolone derivatives having an active leaving group, such that in the presence of an oxidizing agent the or each developer reacts with the or each coupler substantially only at the position having the active leaving group, and (iii) an inorganic peroxygen agent in an amount of from about 0.0001 to about 0.05 moles per 100 g of composition. Alternative compositions may contain higher levels of oxidizing agent provided that they have a pH below 6.

This invention relates to new compositions for colouring hair and to methods of using these compositions in hair colouring processes.

Compositions are well known for providing various colours to hair by dyeing, either for changing a natural hair colour and/or for covering grey hair. Such compositions comprise various aromatic compounds, commonly known as developers (also known as precursors or primary intermediates), together with various other aromatic compounds, commonly known as couplers. These are referred to as oxidative hair colouring agents because they require an oxidising agent for formation of colour.

The developers are generally 1,4-disubstituted benzene compounds, most commonly 1,4-diaminobenzene compounds, and the couplers can also be disubstituted benzene compounds, such as 1,3-disubstituted benzene compounds. The range of structures of couplers is much more varied than that of developers.

In use, the compounds are subjected to oxidising conditions under which the developers and couplers react to form colour. It is generally believed that this occurs by means of a stepwise sequence in which developer molecules are activated by oxidation and react with couplers to form reactive dimers. These then continue reacting to form coloured trimers, which do not react further. It is believed that the monomeric developers and couplers, and to a lesser extent the dimers, diffuse into the hair shaft during the course of the reaction, which is fairly slow. When inside the hair shaft, the dimers react further to form trimers which are too large to diffuse out easily and are thus trapped, colouring the hair.

Standard products normally include several different developers and several different couplers, for instance up to 5 developers and 5 or more couplers. It is generally accepted that 10 to 12 different compounds are required to achieve the full range of colours.

The required oxidising conditions are normally provided by treating the hair with the oxidative hair colouring agents and an oxidising agent. Hydrogen peroxide is the most commonly used oxidising agent.

Typically, hair colouring compositions containing standard oxidative hair colouring agents are formulated at high pH, often from about pH 9 to about pH 12. This high pH is often provided by a source of ammonia. Ammonia has an additional function, in that it acts as a hair swelling agent (HSA). HSA's enhance the oxidising and colouring process by swelling the hair fibers. This aids diffusion of both the oxidising agent and the oxidative hair colouring agents into the hair, which enables faster, more thorough colouring agent oxidation and hair colouring.

Unfortunately, this system, although effective and commercially successful, has various drawbacks.

Firstly, developers can react with each other as well as with couplers and the reactive dimers can react with developers and couplers. Thus the reaction chemistry is undefined and it is not possible to predict with any precision the compounds which will be present in the hair at the end of the colour-forming reaction. The exact composition of the coloured molecules formed in the hair can vary from process to process according to the prevailing conditions. Therefore the colours eventually obtained can vary between applications.

A further drawback is that of fading of colour over time. A contributory factor in fading is lack of wash-fastness. The trimeric coloured molecules produced tend to be soluble in water and in other solvents. Consequently they tend to leach out of the hair after repeated washing and applications of, for instance, hairspray and other hair care products. This leads to gradual fading or changing of the applied colour. The action of factors such as ultraviolet light, combing and perspiration also affects colour.

Use of high levels of hydrogen peroxide also has disadvantages. As well as reacting with oxidative colouring components, hydrogen peroxide can also oxidises the disulphide linkages in the hair. This can lead to undesirable effects in the hair, such as brittleness and hair damage. These problems arise at least in part because it is necessary to use a relatively high concentration of oxidising solution in order to deliver effective oxidation of standard oxidative hair colouring agents, usually between about 20% and about 40% volume of oxygen, ie about 6 to 12 wt % based on concentration in the component as supplied to the consumer which contains oxidising agent. A further problem arises from the fact that the oxidation reaction involves two steps. Thus sufficient oxidising agent must be present in the hair dye composition to induce the two oxidation steps. When hydrogen peroxide is used it is often at a concentration of about 3 wt % in the treatment solution which is applied to the hair. Normal hair colouring processes require extended exposure (from 10 to 60 minutes) of the hair to the colouring composition. Under these harsh, high oxidising agent conditions, hair damage can occur, for instance by oxidation of disulphide linkages in the hair.

The inclusion of high levels of ammonia or other bases can also lead to skin irritation and hair damage. Ammonia and other bases also tend to swell the skin as well as the hair, thus encouraging staining of the skin as well as colouring of the hair. Furthermore ammonia has an undesirable odour and can give rise to lachrymatory effects.

The existence of the high pH conditions which are normally necessary for standard oxidative colouring materials can also lead to hair damage.

These problems have existed with commercial products for many years and have not yet been solved.

GB 1,025,916 discloses certain developers and couplers of different types. It document describes developers which are N,N-disubstituted phenylene diamine derivatives. Three classes of coupler are described. Some phenol-based couplers are said to provide a blue colour, some R—CO—CH₂—COR derivatives are said to provide a yellow colour and some pyrazolone derivatives are said to provide a red colour.

These combinations are advantageous in that the developers do not react with themselves and can react with each coupler in only one way, so that the final chemistry of the dye obtained is closely defined and highly predictable.

GB 1,025,916 describes mixing pairs of couplers, for instance red with blue, blue with yellow, etc so as to obtain shades between the colours which would be obtained with either of the couplers used individually. It describes various examples of dyeing hair using the disclosed developers and couplers. In some examples developer is applied and left for a period of time, followed by application of coupler, which is also left for a period of time and is then followed by application of oxidising agent.

The majority of the examples describe mixing developer, coupler and hydrogen peroxide as oxidising agent and applying the mixture to the hair. This is then left for a period of time, normally 20 minutes, and the hair rinsed. This latter method is the standard method of applying currently commercially available hair dyes containing oxidative hair colouring agents. These are normally supplied in a package containing two bottles. One contains developers and couplers and the other contains oxidising agent. These are mixed before application of the mixture to the hair.

All of the described examples of GB 1,025,916 use the standard oxidative hair colouring conditions of high pH (at least pH 10 in all cases), high ammonia or caustic soda content, and high levels of oxidising agent (about 3 wt % hydrogen peroxide in the solution applied).

Therefore the methods described in GB 1,025,916 still suffer from the problems of hair brittleness and damage and skin irritation and staining found with standard oxidative dyeing systems.

It would be desirable to be able to provide a hair colouring system in which the final colour produced is predictable and controllable but which also reduces hair brittleness and damage and has the potential to reduce skin staining and irritation.

According to a first aspect of the invention we provide a hair colouring composition comprising

(i) one or more developers selected from amino aromatic system capable of being oxidised and thereafter undergoing a single electrophilic attack, and

(ii) one or more couplers selected from

(A) phenols and naphthols having an active leaving group in the Para position relative to the hydroxyl group,

(B) 1,3-diketones containing the group

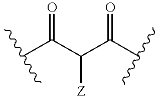

in which Z is an active leaving group, and

(C) compounds containing the group

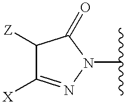

in which Z is an active leaving group, and X is an active leaving group or a non-leaving substituent,

such that in the presence of an oxidising agent the or each developer reacts with the or each coupler substantially only at the position having the active leaving group Z and, if X is an active leaving group, X,

and (iii) inorganic peroxygen oxidising agent in an amount of from about 0.0001 to about 0.05 moles per 100 g of composition.

In the invention the developer is an amino aromatic compound which has a structure such that it is capable of being oxidised by an oxidising agent. The structure is also such that the oxidised developer is capable of undergoing electrophilic attack by one other molecule. In other words, the structure of the developer is such that it reacts substantially only at one position, which is normally an amine. Suitable developers of this type include aminoaromatic systems in which there is only one primary amine group, at which reaction occurs, other amine and other reactive groups being protected by blocking substituents.

The three defined types of coupler are such that in the presence of an oxidising agent the majority couple with the developer at only one position so as to produce only one resulting coloured dimer. Certain couplers of type (C), having an additional active leaving group X, also react at the X position to give a single resulting coloured trimer. In this case also only one type of final coloured molecule is produced from that coupler. The defined developer also reacts only at one position.

Formation of colour is, we believe, by reaction of one or two developer molecules with one coupler molecule to form a coloured dimer or trimer. The dimers and trimers are not reactive and no further reaction takes place. Consequently the formation of colour is extremely efficient. Further, the coloured molecules formed are very pure. With knowledge of the developer and coupler molecules present in the reaction system it is possible to predict closely and accurately the final combination of coloured molecules, and hence the final overall colour, which will be produced. These are significant advantages in comparison with standard oxidative colouring systems.

Further, the coloured molecules formed have significantly reduced water-solubility in the hair in comparison with the trimers formed in standard oxidative colouring systems, which assists in achieving increased wash fastness.

We have also found that the reaction between the defined developer and the defined couplers is potentially very fast and efficient. This gives potential for hair colouring systems which do not require the long exposure times which have up to now been standard in order to produce a deep and lasting colouration.

Additionally, we have found that, surprisingly, it is possible using the defined developers and couplers to provide compositions containing very low levels of inorganic peroxygen oxidising agent which nevertheless give effective and rapid colouration of the hair. As a result, damage to the hair is reduced.

We have also found that with the use of low levels of oxidising agent as defined, the possibility is opened up for use of systems at low pH. This allows for the possibility of reducing levels of ammonia, and even for total removal of ammonia, and consequent reductions in undesirable odour, lachrymatory effects, skin irritation and skin staining and hair damage.

A further benefit of the composition of the invention lies in the fact that reduction in levels of oxidising agent can lead to a more economical product.

Some suitable coupler molecules (A), (B) and (C) are known from the field of photography, as are some suitable developers. When they have reacted with a developer molecule the couplers (A) give a cyan colour, the couplers (B) give a yellow colour and the couplers (C) give a magenta colour.

Each coupler contains a moiety of a specific formula which is such that it has an active leaving group Z at a defined site. By an “active leaving group” we mean any group which can be removed (under the conditions prevailing during the hair-dyeing process) so that the developer reacts at that position in the coupler molecule. The bond formed between the coupler and developer molecule is thus formed at the site of the active leaving group. Examples of active leaving groups are H, PhO, Cl, Br, alkoxy (RO) such as phenoxy PhO, and RS—in which R is alkyl or aryl, but any leaving group which leaves during the reaction so as to allow coupling between developer and coupler is suitable.

If X is an active leaving group it may be any of those listed above for Z.

Couplers (A) give a cyan colour. The particular shade or intensity of colour can be varied by varying the substituents of the phenol or naphthol molecule. It has an active leaving group para to the OH group. This may be an active proton, ie the aromatic ring is unsubstituted in the para position and other substituents on the ring are not such as to reduce the reactivity at this position.

Generally couplers (A) have the formula I, as follows:

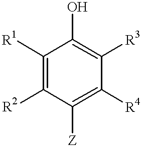

in which Z is H or another active leaving group. Preferably Z is H.

R¹, R^{2,} R³ and R⁴ are, independently, H, OH, —CO₂H, —CO₂R, F, Cl, Br, —CN, —NO₂—, CF₃, cycloalkyl, alkenyl, cycloalkenyl, aryl, alkaryl, aralkyl, —NH₂, —NHR, —NHCOR, —NR₂, —NHCOR, —R′NHCOR, —CONHR, R′CONHR, —R′OH, —SO₂R, SO₂NHR, —R′SO₂R, —R′SO₂NHR, —SO₃H, —OR, —R′OR or —COR, in any of which R is H, alkyl, cycloalkyl, alkenyl, cycloalkenyl, aryl, alkaryl or aralkyl, and R′ is alkylene, alkenylene, cycloalkylene, cycloalkenylene, arylene, alkarylene or aralkylene, or substituted versions of any of these. Additionally, R¹ and R² may together form a substituted or unsubstituted cycloalkyl, cycloalkenyl or aryl group. Substituting groups include OH, —OR, Cl, Br, F, —CO₂H, —CO₂R, —NH₂ and —COR.

In this specification, unless otherwise stated alkyl and alkenyl are usually C₁₋₈, often C₁₋₄, cycloalkyl and cycloalkenyl are usually C₅₋₈, often C₆, aryl or ar- is usually phenyl or naphthyl and the alk-moiety in alkaryl is usually C₁₋₆, often C₁₋₄.

For coupler (A) it is preferred that when R¹, R², R³ or R⁴ is alkyl it is selected from methyl, ethyl, n-propyl, i-propyl and t-butyl. When R is alkyl it is preferably one of these groups and when R¹ is alkylene it is preferably derived from one of these groups.

Substituents R¹ and R² may form a second benzene ring, so that coupler (A) is a naphthol derivative of the formula II, as follows:

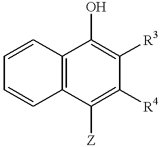

In this case R³ and R⁴ are preferably H and the developer is α-naphthol.

In other suitable couplers (A) of formula II, R⁴ is H and R³ is

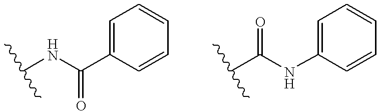

Suitable couplers (A) thus have the formula III or IV, as follows:

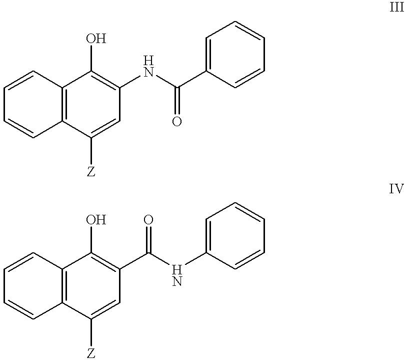

Coupler (A) can be a naphthol having no solubilising substituents (other than Z), in particular no —COOH or —OH substituents. Naphthols are preferably unsubstituted.

We find that couplers of this formula II, in particular when R³ and R⁴ are H, and especially when Z is H, have a particularly advantageous combination of properties for improving wash fastness whilst allowing rapid colouring, when the hair to be coloured has been damaged, for instance by perming or bleaching. We believe this is because their molecules have a structure such that as monomers they are small enough to diffuse easily into the hair shaft (which, when damaged, is rather porous) but as dimers they are trapped within the hair shaft. Further, their water-solubility is low enough that they are not easily washed out during subsequent hair treatment processes.

Preferred couplers have the formula I in which R¹, R², R³ and R⁴ are independently selected from OH, H, methyl, ethyl, n-propyl, i-propyl, t-butyl, NH₂, —CO₂H, and —COR. In these preferred couplers (A) Z is H.

A preferred coupler of this preferred type is 3-amino phenol.

We find that these preferred couplers show particularly good performance on damaged hair. They demonstrate good colour uptake and good wash fastness.

In all of the above formulae Z is any active leaving group. Suitable examples are H, PhO, Cl and Br but any other groups which react similarly (under the conditions of the hair-dyeing reaction) may be used. If Z is PhO, Cl or Br the reactivity of the coupler can tend to be increased in comparison with couplers in which Z is H.

In any of the above formulae, unless otherwise stated, the defined groups may also contain any non-interfering substituent, that is any group which does not hinder the coupling reaction between developer and coupler. In particular, phenyl and naphthyl groups may be substituted. Suitable non-interfering substituents include CO₂H, CH₃, SO₂ NHCH₃, SO₃H, C₁₋₃ alkyl such as ethyl or propyl and CONHR in which R is preferably C₁₋₃ alkyl. Alkyl and CONHR substituents have the advantage that the solubility of the final coloured molecule is reduced. Phenyl groups may contain one or more substituents which are the same or different. If phenyl groups are substituted, mono substitution is preferred. Preferably the groups are unsubstituted unless otherwise stated.

Yellow couplers (B) contain the 1,3-diketone group

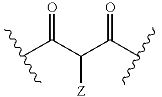

in which Z is an active leaving group. Generally, they have the formula V as follows:

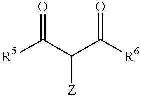

in which R⁵ and R⁶ are, independently, H, alkyl, cycloalkyl, alkenyl, cycloalkenyl, aryl, alkaryl, aralkyl, —R′NHCOR, —RCONHR, —ROH, —R′SO₂R, —R′CO₂NHR, —NHCOR, —NR₂, —NHR, —NH₂, —R′OR and —OR. In these groups R can be H, alkyl, cycloalkyl, alkenyl, cycloalkenyl, aryl, alkaryl or aralkyl and R′ is alkylene, cycloalkylene, alkenylene, cycloalkenylene, arylene, alkarylene or aralkylene. Substituted versions of any of these can be used. Suitable substituents include OH, —OR, Cl, Br, F, —CO₂H, —CO₂R, —NH₂ and —COR.

In some preferred formulae, at least one of R⁵ and R⁶ contains an aryl group.

Some couplers (B) are of the formula VI as follows:

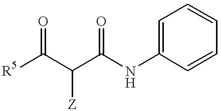

In this formula R⁵ can be for instance methyl, phenyl, t-butyl or N(CH₃)CH₂CH₂OH. When R⁵ is tertiary butyl advantages arise from the fact that the coloured dimer produced has particularly good resistance to breakdown by light. R⁵ may also be phenyl. In formula VI it can be preferred that the N-phenyl does not contain solubilising substituents. In particular it can be preferred that it does not contain —COOH or —OH substituents. Preferably the N-phenyl is unsubstituted. In formulae in which R⁵ is also phenyl it can be preferred that the R⁵ phenyl is free of solubilising substituents, in particular free of —COOH and —OH substituents and is most preferably unsubstituted.

We find that couplers of the formula VI have a particularly advantageous combination of properties for improving wash fastness whilst allowing rapid colouring, in particular for damaged hair. We believe this is because their molecules have a structure such that as monomers they are small enough to diffuse easily into the porous, damaged hair shaft but as dimers they are trapped within the hair shaft. Further, their solubility is such that they are not easily washed out during subsequent hair treatment processes.

In other preferred couplers (B) R⁵ is methyl, ethyl, n-propyl, i-propyl, t-butyl or phenyl (especially methyl) and R⁶ is NR₂ in which the R groups are the same or different and can be R as discussed above, in particular methyl, ethyl, n-propyl, i-propyl, t-butyl or phenyl (especially ethyl).

In other suitable preferred couplers (B) R⁵ and R⁶ are, independently, short chain (C₁₋₄) alkyl such as methyl, ethyl, i-propyl, n-propyl or t-butyl or short chain (C₁₋₄) alkoxy, such as methoxy or ethoxy. In particular, R⁵ is C₁₋₄ alkyl (especially methyl) and R⁶ is C₁₋₄ alkyl (especially methyl) or C₁₋₄ alkoxy (especially methoxy).

In these formulae alkyl groups can advantageously be hydroxylated, to produce for instance hydroxymethyl (usually 2-hydroxyethyl), hydroxyethyl, hydroxypropyl or hydroxybutyl.

Couplers (B) of these latter types are particularly advantageous for the coloration of undamaged as well as damaged hair. On undamaged hair they show fast colour uptake without loss of wash fastness. They also show good wash fastness on damaged hair.

In formulae V and VI, Z may be any of the leaving groups indicated for Z in coupler (A) above. Preferably Z is H.

In any of the above formulae, unless otherwise stated, the defined groups may also contain any non-interfering substituent, that is any group which does not hinder the coupling reaction between developer and coupler. In particular, phenyl and naphthyl groups may be substituted. Suitable non-interfering substituents include CO₂H, CH₃, SO₂ NHCH₃, SO₃H, C₁₋₃ alkyl such as ethyl or propyl and CONHR in which R is preferably C₁₋₃ alkyl. Alkyl and CONHR substituents have the advantage that the solubility of the final coloured molecule is reduced. Phenyl groups may contain one or more substituents which are the same or different. If phenyl groups are substituted, mono substitution is preferred. Preferably the groups are unsubstituted unless otherwise stated.

Couplers (C) are pyrazolone derivatives, that is they contain the group

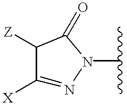

in which Z is an active leaving group and X is an active leaving group or a non-leaving substituent.

Normally X is a non-leaving substituent and they are of the formula VII, as follows:

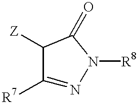

in which R⁷ may be H, —OH, —CO₂H, —CO₂R, F, Cl, Br, —CN, —NO₂, CF₃, alkyl, cycloalkyl, alkenyl, cycloalkenyl, aryl, alkaryl, aralkyl, —NH₂, —NHR, —NR₂, —NHCOR, —R′NHCOR, —CONHR, —RCONHR, —R′OH, —SO₂R, —SO₂NHR, —R′SO₂R, —R′SO₂NHR, —SO₃H, —OR, —R′OR or —COR. R⁸ can be H, alkyl, alkenyl, cycloalkyl, cycloalkenyl, aryl, alkaryl, aralkyl, —R′NHCOR, —RCONHR, —R′OH, —R′SO₂R, —R′SO₂NHR or —R′OR. R is H, alkyl, cycloalkyl, alkenyl, cycloalkenyl, aryl, alkaryl or aralkyl, and R′ is alkylene, cycloalkylene, alkenylene, cycloalkenylene, arylene, alkarylene or aralkylene (or substituted versions of any of these). Suitable substituting groups include OH, —OR, Cl, Br, F, —CO₂H, —CO₂R, —NH₂, and —COR.

For instance R⁷ can be H or methyl. It may alternatively be —NHR or —NHCOR in which R is alkyl, cycloalkyl, alkenyl, cycloalkenyl, aryl, alkaryl or aralkyl.

Advantageously R⁷ is H, lower (C₁₋₄) alkyl such as methyl, ethyl, n-propyl, i-propyl or t-butyl, or substituted or unsubstitited phenyl, in particular H, methyl or methylphenyl.

R⁸ is advantageously H, lower (C₁₋₄) alkyl such as methyl, ethyl, n-propyl, i-propyl or t-butyl, or substituted or unsubstituted phenyl. When it is phenyl, it can be preferred that it is a phenyl free of solubilising substituents, in particular —COOH and —OH substituents. m-SO₃H and p-SO₃H substituents can be used. When R⁸ is phenyl it can preferably be unsubstituted. R⁸ can also preferably be H, phenyl or methyl.

We find that couplers of these latter formulae have a particularly advantageous combination of properties for improving wash fastness whilst allowing rapid colouring, especially on undamaged hair. We believe this is because their molecules have a structure such that as monomers they are small enough to diffuse easily into the undamaged hair shaft but as dimers they are trapped within the hair shaft. Further, their solubility is such that they are not easily washed out during subsequent hair treatment processes.

Suitable couplers (C) have the formula VIII, as follows:

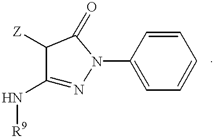

in which R⁹ is preferably

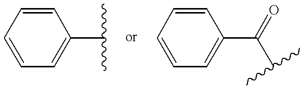

In formulae VII and VIII, Z may be any of the leaving groups indicated for Z in couplers (A) and (B) above.

In any of the above formulae, unless otherwise stated, the listed groups may also contain any non-interfering substituent, that is any group which does not hinder the coupling reaction between developer and coupler. In particular, phenyl and naphthyl groups may be substituted. Suitable non-interfering substituents include CO₂H, CH₃, SO₂, NHCH₃, SO₃H, C₁₋₃ alkyl such as ethyl or propyl and CONHR in which R is preferably C₁₋₃ alkyl. Alkyl and CONHR substituents have the advantage that the solubility of the final coloured molecule is reduced. Phenyl groups may contain one or more substituents which are the same or different. If phenyl groups are substituted, mono substitution is preferred. Preferably groups are unsubstituted unless otherwise stated.

Specific examples of couplers (A) include α-naphthol, 3-aminophenol and the compounds having the following structural formulae:

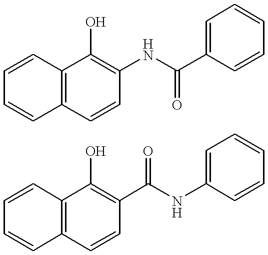

Specific examples of couplers (B) include benzoylacetanilide, acetoacetanilide, N,N-diethyl and N,N-dimethyl acetoacetamide and the compounds of the formulae

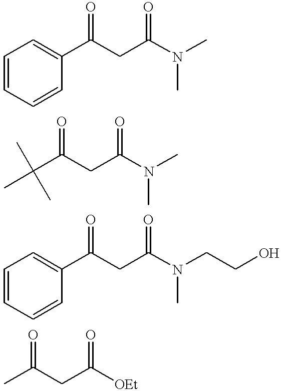

Specific examples of couplers (C) include the pyrazolone of structural formula:

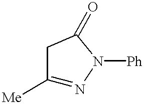

and compounds having the same formula except that Ph is replaced by H or methyl and/or Me is replaced by H, and compounds having the following structural formulae:

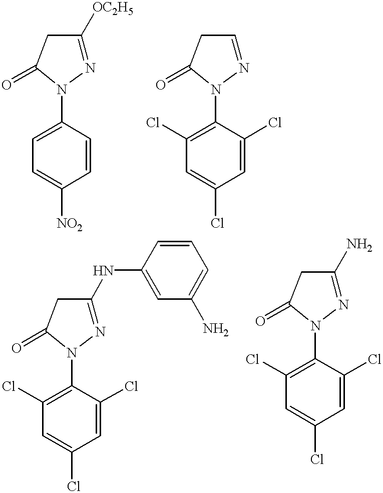

Any of the couplers discussed above may also be used in the salt form, for instance sulphate, phosphate and hydrochloride, particularly sulphate or hydrochloride.

Compounds containing free amine groups are preferably used in the form of their salt. The salt form of such compounds forms a powder and is often more stable than the free base form.

The solubility of the couplers and their structure should be selected so that they are sufficiently soluble to be formulated into a suitable application form, which may be aqueous, and of solubility and size such that they can diffuse sufficiently rapidly into the hair shaft. We find as a general rule that couplers of low solubility and large molecular size tend to diffuse sufficiently rapidly into damaged hair. We find that for undamaged hair smaller, more soluble molecules are particularly suitable.

In addition, the final coloured molecule produced must be of size and solubility that it does not easily wash out of the hair shaft. Again, we find that larger, more insoluble coloured molecules give better wash fastness in damaged hair and smaller, more soluble molecules give good wash fastness in undamaged hair.

The composition of the invention may contain any one, or more, of the couplers (A), (B) and (C) in combination with the defined developer and the antioxidant. A particular advantage of the use of these particular couplers is that it is possible to obtain the full range of colours using just three specific types of coupler and one type of developer. Preferably the composition contains at least two out of the three types of coupler. In particular it contains at least one coupler (B) or (C). More preferably it contains at least one coupler of each type (A), (B) and (C). In some preferred compositions not more than two or even only one compound of any or all of the types (A), (B) and (C) is included.

This allows the couplers to be supplied in the form of a mixture having the required amounts of each type of coupler to develop whichever colour is desired. Alternatively, it also allows supply of the coupler materials separately packaged so that the consumer can control the eventual colour which is obtained by mixing the correct amounts of each coupler to form the composition.

Usually coupler is present in the composition in a total amount of from 0.001% to 5 or 10% by weight based on total weight of composition applied to the hair. Preferably total amounts of coupler are at least 0.01 wt %, often at least 0.1 or 1%. Preferably they are not more than 6%, and in some compositions preferred amounts are up to 3%, for instance not more than 2.5%.

Couplers of types (A) and (C) can be used in particularly low amounts. For instance couplers of type (A) may be used in amounts, by weight based on total weight of composition applied to the hair, of from 0.001 to 1%, preferably 0.004 or 0.005 to 0.5%, for instance not more than 0.05 wt %. Couplers of type (C) can be used in amounts of from for instance 0.01 to 2 or 4%, preferably 0.03 to 3% and in some preferred compositions up to 1 or 0.5%. Couplers of type (B) are often used in larger amounts, for instance from 0.05 to 3 or 4 wt %, for instance 0.1 to 2 or 3 wt %.

The developer is an amino aromatic compound capable of being oxidised and undergoing a single electrophilic attack in the oxidised state. For instance it may be an aromatic system containing a single primary amine substituent.

The developer is such that it reacts substantially at only one position (normally the amine position). In some cases the structure of the developer may be such that it is possible that it reacts with other developer molecules, but it reacts preferentially with coupler molecules. Preferably the structure of the developer is such that it undergoes substantially no reaction with other developer molecules.

Suitable developers include o-nitro and p-nitro α-naphthylamines of the formulae

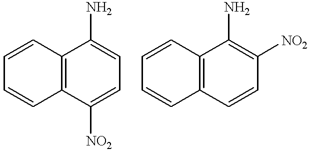

Other suitable developers include o- and p-nitrophenylamines H₂N—Ph—NO₂, N,N-disubstituted o-phenylene diamines and, N,N-disubstituted p-phenylene diamines.

The developer can be an N,N-disubstituted p-phenylene diamine. These developers have an amine group protected by disubstitution and react only at the primary amine group. In this case it normally has the formula IX, as follows:

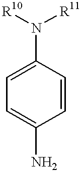

in which R¹⁰ and R¹¹ are each independently H, alkyl, cycloalkyl, alkenyl, cycloalkenyl, aryl, alkaryl, aralkyl, —R′NHCOR, —RCONHR, —R′OH, —R′SO₂R, —R′SO₂NHR OR —R′OR in which R is alkyl, cycloalkyl, alkenyl, cycloalkenyl, aryl, alkaryl, aralkyl, and R′ is alkylene, cycloalkylene, alkenylene, cycloalkenylene, arylene, alkarylene or aralkylene, or substituted versions of any of these. Suitable substituting groups include OH, —OR, Cl, Br, F, —CO₂H, —CO₂R, —OR and —COR. Alternatively, R¹⁰ and R¹¹ may together form a substituted or unsubstituted cycloalkyl, cycloalkenyl or aryl ring.

Preferably R¹⁰ and R¹¹ are, independently, C₁4 alkyl, preferably —CH₃, —CH₂CH₃ or i-propyl; C₁₋₃ hydroxyalkyl, preferably —CH₂CH₂OH; alkylene alkoxy, preferably ethylmethoxy (—CH₂CH₂OCH₂); or R¹²SO₂NHR¹² or R¹²NHSO₂R¹² in which R¹² is C₁₋₃ alkyl, for instance —CH₂CH₂SO₂NHCH₃ or —CH₂CH₂NHSO₂CH₃.

Particularly preferred developers of the above formula IX are those in which R¹⁰ and R¹¹ are both —CH₂CH₃ or R¹⁰ is —CH₂CH₃ and R¹¹ is —CH₂CH₂NHSO₂CH₃. The latter R¹¹ substituent is believed to contribute to dermatological compatibility. In other suitable developers R¹⁰ is ethyl and R¹¹ is hydroxyethyl; or R¹⁰ is ethyl and R¹¹ is —CH₂CH₂OCH₃; or R¹⁰ is selected from H, methyl, ethyl, and propyl and R¹¹ is selected from methyl, ethyl and propyl.

In general the developer may be selected from compounds having the general formula X:

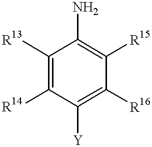

The group Y is a blocking group which ensures that reaction (under the conditions of dyeing the hair) takes place only at the primary amine group. The group Y for instance can be —NR¹⁰R¹¹ (as in formula IX above) Other suitable Y groups include —NO₂, —CO₂H, —CO₂R, —COR and OH. R is as defined above for formula IX.

In an alternative developer formula, the blocking group Y is in the ortho position relative to the amino group, giving the following formula XI.

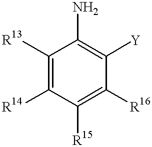

Thus the group Y is positioned so that the developer undergoes only one reaction, at the primary amine group, under the conditions of the reaction.

R¹³, R¹⁴, R¹⁵ and R¹⁶ can each be, independently, any of the groups listed for R¹ to R⁴ above. R¹³ and R¹⁴ together and/or R¹⁵ and R¹⁶ together, may form a substituted or unsubstituted cycloalkyl, cycloalkenyl or aryl ring.

Preferably R¹³ to R¹⁶ are, independently, H, methyl, ethyl, n-propyl, i-propyl, F, Cl, OH, —CO₂H, —CO₂R or —COR.

In any of the above formulae, unless otherwise stated, the listed groups may also contain any non-interfering substituent, that is any group which does not hinder the coupling reaction between developer and coupler. In particular, phenyl and naphthyl groups may be substituted. Suitable non-interfering substituents include CO₂H, CH₃, SO², NHCH₃, SO₃H, C₁₋₃ alkyl such as ethyl or propyl and CONHR in which R is preferably C₁₋₃ alkyl. Alkyl and CONHR substituents have the advantage that the solubility of the final coloured molecule is reduced. Phenyl groups may contain one or more substituents which are the same or different. If phenyl groups are substituted, mono substitution is preferred. Preferably the groups are unsubstituted unless otherwise stated.

A preferred developer has the following formula XII, as follows:

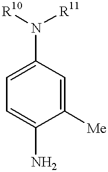

Specific examples of developers of the invention are those of the following structural formulae:

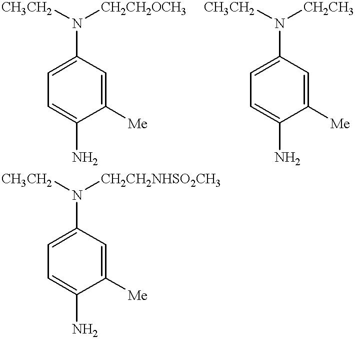

These are suitable especially for colouring of damaged hair.

Additional developers of the invention are:

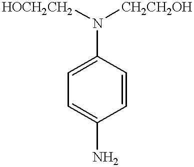

which tends to fade more rapidly than certain others, and

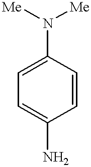

which is highly reactive.

Examples of further preferred developers, which are particularly suitable for coloration of undamaged hair, are 2,6-dichloro-p-aminophenol, 3-chloro-p-aminophenol, 2-chloro-p-aminophenol 2,3-dichloro-p-aminophenol and 3,5-methyl-p-aminophenol.

The derivatives above include salts, for instance sulphate, phosphate and hydrochloride, particularly sulphate or hydrochloride. Salts are normally formed with the amine groups. The preferred developer in which R¹⁰ is —CH₂CH₃ and R¹¹ is CH₂CH₂NHSO₂CH₃ is often provided in salt form as a stable powder (more stable than the free base form). We have found that this salt forms such that it contains 3 moles of salt molecule to 2 moles of the free base molecule. A further useful salt is the hydrochloride salt of developers such as 2,6-dichloro-p-aminophenol.

Developer is often included in the composition in amounts of from 0.01 to 5 or 7% by weight based on total composition applied to the hair. Preferred amounts of developer are from 0.3 to 2 or 4%, preferably 0.4 to 1.5 or 3%.

We find that if the developer and coupler compounds are sufficiently soluble in the composition, under the conditions of application to the hair, they will diffuse sufficiently rapidly into the hair shaft. However, the coloured molecules produced should be of sufficiently low solubility that they resist washing out of the hair.

For both developers and couplers the solubility properties can be important. The developer and coupler compounds themselves should have solubility such that they can be formulated in appropriate concentrations. For application at high pH preferably have solubility of at least 10, more preferably at least 15 and most preferably at least 20 g/100 ml deionised water at pH about 10 and 25° C. They may have solubility at least 25 g/100 ml, and even up to 50 or 80 g/100 ml but normally it is not more than 30 g/100 ml.

The developer and coupler compounds are also generally such that the solubility of the final coloured dimer (or trimer if produced) is low under normal hair conditions and, especially, conditions of washing. Thus solubility (at pH about 8) of the final coloured molecule is preferably below 5 g/100 ml deionised water at 25° C., in particular below 2 or 1 g/100 ml and most preferably below 0.5 g/100 ml or even below 0.2 g/100 ml.

In compositions which are to be applied at high pH (for instance above pH 10), an indication of solubility can sometimes be given by pKa. Thus if one or more of the developers and couplers, in particular the developers, has an ionisable group which is substantially ionised at a pH of above 9, preferably above pH 10, this is an indicator of solubility at about pH 10. However, in the final coloured molecule and at the pH in the hair shaft (which is usually about pH 5.5 to 6) it becomes non-ionised. This gives an indication that under normal conditions it has reduced solubility. This can often be achieved by providing at least one group which has a pKa of from 8 to 12 (and is thus ionised above that pH) in a developer or coupler molecule and which on reaction to form a final coloured molecule also has pKa of from 8 to 12 (and is thus non-ionised at below that pH). Solubility can be affected by various factors but pKa can be a good indicator of likely solubility in some cases.

We find that an advantage of the colouring compounds of the invention is that they can give even coloration and fade resistance on both damaged and undamaged hair, This is particularly useful in cases where the hair has been dyed once and then allowed to grow so that undyed, undamaged hair appears. On redyeing, the undamaged hair and the faded, dyed, damaged hair must both be coloured and show even fade resistance. It is particularly important to be able to provide colour, wash fastness and fade resistance to damaged (eg bleached and/or permed and/or previously dyed) hair. An advantage of the composition of the invention is that the full range of colours can be achieved using a very small number of compounds, in contrast to standard oxidative dyeing systems. Preferably only one or two, in particular only one, developer compound is used. In particular it is preferred that this is used in combination with not more than three, preferably only one or only two, compounds of any of the types (A), (B) and (C).

All of these couplers and developers can be classed as “oxidative” colouring agents, since they require the presence of an oxidising agent to initiate their reaction. Preferably these are the only oxidative colouring agents present in the composition and less than 0.1 wt %, in particular less than 0.08 wt % or less than 0.05 wt %, and especially substantially no oxidative colouring agents are included which are not of the types (i) and (A), (B) and (C), preferably the formulae discussed above.

It is preferred that the composition contains not more than 0.1 wt %, preferably not more than 0.08 wt % or not more than 0.05 wt % and especially substantially no oxidative dye materials which are capable of undergoing reaction more than once (under the oxidising conditions of the dyeing reaction).

It is preferred that not more than 0.1 wt %, preferably not more than 0.08 wt %, most preferably not more than 0.05 wt %, and especially substantially none, of any material which can react with its own molecules is included, Preferably the total of such materials is not more than these values.

Other colouring agents such as vegetable dyes can be included, but it is preferred that no non-oxidative dyes are present and indeed preferably no other colouring components are included than the developer (i) as defined and couplers (A), (B) and (C). That is, in the hair dye composition the colouring components consist essentially of developer (i) and couplers (A), (B) and/or (C). Trivial amounts of other colouring components can of course be included provided they do not significantly influence the final colour.

The compositions of the invention give particularly good colour uptake and fade resistance when used to colour hair which has been damaged, eg. by bleaching, previous colouring or, especially, perming.

An advantage of the composition of the invention is that the full range of colours can be achieved using a very small number of compounds, in contrast to standard oxidative dyeing systems. Preferably only one or two, in particular only one, developer compound is used. In particular it is preferred that this is used in combination with not more than three, preferably only one or only two, compounds of any of the types (A), (B) and (C).

Inorganic peroxygen oxidising agent is present in the composition in an amount of from about 0.0001 to about 0.05 moles per 100 g of composition.

Amounts of about 0.05 moles per 100 gram of composition and below are low enough that the hair damaging effects of high levels of inorganic peroxygen oxidising agents such as hydrogen peroxide are reduced. Amounts of about 0.0001 moles per 100 gram of composition and above remain high enough to ensure that an oxidising effect is exerted on the colouring agents so as to produce the desired coloured molecules.

Preferably it is present in an amount of not more than about 0.04, more preferably not more than about 0.03 moles per 100 g of composition. Amounts of about 0.03 moles per 100 g of composition can be particularly effective. Often the amount of inorganic peroxygen oxidising agent is at least 0.0003 moles per 100 g of composition, preferably at least 0.001 or 0.002 moles.

Preferred compositions of the invention comprise inorganic peroxygen oxidising agent in an amount of from about 0.003 to about 1.5 wt %, more preferably not more than about 1.2 wt %, most preferably not more than about 1.0 wt %. Preferred compositions often contain inorganic peroxygen oxidising agent in an amount of about 1 wt %. Preferably the amount of inorganic peroxygen oxidising agent is at least 0.005 wt %, preferably at least 0.01 wt %.

Preferred oxidising agents of all types are water-soluble. Water-soluble oxidising agents as defined herein are those which have a solubility to the extent of about 10 g in 1,000 ml of deionised water at 25° C. (“Chemistry”, C. E. Mortimer, 5th Edition, page 277).

Suitable inorganic oxidising agents include hydrogen peroxide, sodium periodate, sodium perbromate and sodium peroxide, and inorganic perhydrate salt oxidising compounds such as the alkali metal salts of perborates, percarbonates, perphosphates, persilicates, and persulphates. Inorganic perhydrate salts may be incorporated as monohydrates, tetrahydrates etc. Mixtures of two or more of such inorganic peroxygen oxidising agents can be used if desired. Alkali metal bromates and iodates are suitable, bromates being preferred.

Hydrogen peroxide is highly preferred for use in the invention. We have found that the invention has particular benefits where the oxidising agent is hydrogen peroxide, because this is a very effective oxidising agent but can lead to greater problems of damage than can other oxidising agents. Consequently the ability to reduce its levels carries particular benefits.

The total oxidising agent in the composition preferably consists essentially only of inorganic oxidising agent. However, the total amount of oxidising agent may include other oxidising agents including preformed organic peroxyacid oxidising agents and other organic peroxides such as urea peroxide, melamine peroxide and mixtures of any of these.

Suitable preformed organic peroxyacid oxidising agents have the general formula R³⁰C(O)OOH, in which R³⁰ is selected from saturated or unsaturated, substituted or unsubstituted, straight or branched chain, alkyl, aryl or alkaryl groups with from 1 to 14 carbon atoms.

One class of organic peroxyacid compounds suitable for use in the invention is that of the amide substituted compounds of the following general formulae XII and XIV:

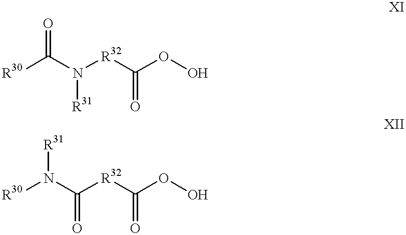

wherein R³⁰ is a saturated or unsaturated alkyl or alkaryl group or an aryl group, having from 1 to 14 carbon atoms, R³² is a saturated or unsaturated alkyl or alkaryl group, or an aryl group, having from 1 to 14 carbon atoms and R³¹ is H or a saturated or an unsaturated alkyl or alkaryl group, or an aryl group, having from 1 to 10 carbon atoms. Amide substituted organic peroxyacid compounds of this type are described in EP-A-170,386.

Other suitable organic peroxyacid oxidising agents include peracetic, pernanoic, nonylamidoperoxycaproic acid (NAPCA), perbenzoic, m-chloroperbenzoic, di-peroxy-isophthalic, mono-peroxyphthalic, peroxylauric, hexanesulphonyl peroxy propionic, N,N-phthaloylamino peroxycaproic, monoper succinic, nonanoyloxybenzoic, dodecanedioyl-monoperoxybenzoic, nonylamide of peroxyadipic acid, diacyl and tetraacylperoxides, especially diperoxydodecanedioic acid, diperoxytetradecanedioic acid and diperoxyhexadecanedioic acid and derivatives thereof. Mono- and diperazelaic acid, mono- and diperbrassylic acid and N-phthaloylaminoperoxicaproic acid and derivatives thereof are also suitable for use in the invention.

Preferred peroxyacid materials are selected from peracetic and pernanoic acids and mixtures thereof.

If non-inorganic oxidising agents are included, these are preferably present in amounts of from about preformed organics peroxyacids 0.0001 to 0.1 mol per 100 g of compositions, preferably from about 0.001 to 0.05 mol, more preferably from about 0.003 to 0.04 mol, especially from about 0.004 to 0.03 mol/100 g.

The preformed organic peroxyacid oxidising agent, where present, is preferably present at a level of from about 0.01% to about 8%, more preferably from about 0.1% to about 6%, most preferably from about 0.2% to about 4%, and especially from about 0.3% to about 3% by weight of the hair colouring composition.

If both inorganic oxidising agent and preformed organic peroxyacid oxidising agent are used, the weight ratio of inorganic peroxygen oxidising agent to preformed organic peroxyacid is preferably in the range from about 0.0125:1 to about 500:1, more preferably from about 0.0125:1 to about 50:1.

If additional organic peroxides are used, suitable amounts are from about 0.01% to about 3%, preferably from about 0.01% to about 2%, more preferably from about 0.1% to about 1.5% and most preferably from about 0.2% to about 1% by weight of composition.

The compositions of the invention may be formulated so that they have a high pH, that is above pH 6.1 or 6.5 and in particular above pH 7 or 8, especially about pH 9 to about pH 12. The compositions can contain ammonia (or other base) as a hair swelling agent, especially if they are formulated at high pH. If present, ammonia (or other base) is included in the composition in amounts of preferably at least 0.01 wt %, more preferably at least 0.05 wt %, in particular from 0.1 to 3 wt % based on total composition applied to the hair, for instance 0.2 or 0.4 to 1 or 2 wt %.

Such systems are particularly effective for hair colouring systems in which the aim is to dye grey hairs. The low levels of oxidising agent mean that the coloured hairs are not damaged, but the presence of ammonia encourages swelling of the grey hairs, which are generally accepted to be more difficult to colour than coloured hair, and thus encourages diffusion of the colouring materials and oxidising agent into the grey hairs.

They are also effective for imparting dark colours to light or blonde undamaged hair. This does not require bleaching (which is often effected in known systems using high levels of oxidising agent), but does benefit from the swelling effect of ammonia on undamaged hair, which is also accepted as being more difficult to colour than damaged hair.

The composition of the invention is particularly suitable, however, for the formulation of compositions having a low pH. Preferably therefore the composition comprising developers (i), couplers (A), (B) and/or (C) and oxidising agent has a pH in the range of from about 1 to about 6. Preferably these compositions contain low levels (eg below 0.5 or 0.3 wt %) of ammonia, or other base and are particular preferably substantially free of ammonia or other base.

We have found that low pH compositions are far milder to the hair and skin than standard high pH compositions. Skin irritation and staining are reduced, together with odour and lachrymatory effects. Surprisingly they also give effective oxidation of the colouring agents and colouring of the hair.

Low pH hair colouring systems containing standard oxidative colouring agents are described in our copending application number 9626713.3.

In the low pH compositions of the invention the pH is preferably from about 1.5 to about 5.8, more preferably from about 1.8 to about 5.5, most preferably from about 2 to about 5 and especially from about 3.5 to about 4.5.

It is also preferred that the pH of a composition containing only the developer (i) and couplers (A), (B) and/or (C) has a pH in the range of from about 1 to about 6, preferably from about 1.5 to about 5.8, more preferably from about 1.8 to about 5.5, most preferably from about 2 to about 5 and especially from about 3.5 to about 4.5. We find that at these low pHs the developers and couplers of the invention have a greater storage stability.

The compositions may contain one or more optional buffering agents. Several different pH modifiers can be used to control the pH of the final composition or any constituent part thereof.

This pH adjustment can be effected to achieve low pH by using well known acidifying agents in the field of treating keratinous fibers, and in particular human hair, such as inorganic and organic acids such as hydrochloric acid, tartaric acid, citric acid, succinic acid, phosphoric acid and carboxylic or sulphonic acids such as ascorbic acid, acetic acid, lactic acid, sulphuric acid, formic acid, ammonium sulphate and sodium dihydrogenphosphate/phosphoric acid, disodium hydrogenphosphate/phosphoric acid, potassium chloride/hydrochloric acid, potassium dihydrogen phthalate/hydrochloric acid, sodium citrate/hydrochloric acid, potassium dihydrogen citrate/hydrochloric acid, potassium dihydrogencitrate/citric acid, sodium citrate/citric acid, sodium tartarate/tartaric acid, sodium lactate/lactic acid, sodium acetate/acetic acid, disodium hydrogenphosphate/citric acid and sodium chloride/glycine/hydrochloric acid, succinic acid and mixtures thereof.

Examples of alkaline buffering agents for high pH compositions are ammonium hydroxide, ethylamine, dipropylamine, triethylamine and alkanediamines such as 1,3-diaminopropane, anhydrous alkaline alkanolamines, such as mono or di- ethanolamine, preferably those which are completely substituted on the amine group such as dimethylaminoethanol, polyalkylene polyamines such as diethylenetriamine or a heterocyclic amine such as morpholine as well as the hydroxides of alkali metals, such as sodium and potassium hydroxide, hydroxides of alkali earth metals, such as magnesium and calcium hydroxide, basic amino acids such as L-argenine, lysine, alanine, leucine, iso-leucine, oxylysine and histidine and alkanolamines such as dimethylaminoethanol and aminoalkylpropanediol and mixtures thereof. Also suitable for use herein are compounds that form HCO₃⁻ by dissociation in water (hereinafter referred to as ‘ion forming compounds’). Examples of suitable ion forming compounds are Na₂CO₃, NaHCO₃, K₂CO₃, (NH₄)₂CO₃, NH₄HCO₃, CaCO₃ and Ca(HCO₃) and mixtures thereof.

Preferred for use herein as buffering agents are organic and inorganic acids having a first pKa below pH 6, and their conjugate bases. As defined herein, first pKa means, the negative logarithm (to the base 10) of the equilibrium constant, K, where K is the acid dissociation constant. Suitable organic and inorganic acids for use herein are: aspartic, maleic, tartaric, glutamic, glycolic, acetic, succinic, salycilic, formic, benzoic, malic, lactic, malonic, oxalic, citric, phosphoric acid and mixtures thereof. Particularly preferred are acetic, succinic, salycilic and phosphoric acids and mixtures thereof.

It is also preferred that a composition containing a level of oxidising agent of from about 0.0001 to about 0.05 moles per 100 g of that composition, without the developer and couplers, also has a pH in the range of from about 1 to about 6, preferably from about 1.5 to about 5.8, more preferably from about 1.8 to about 5.5, most preferably from about 2 to about 5 and especially from about 3.5 to about 4.5.

There are various ways in which the composition of the invention can be supplied. It is normally supplied in the form of a hair colouring kit containing the components of the composition in two or more separately packaged components. The consumer mixes the components as instructed to form the composition which is then applied to the hair.

One advantage of the invention which we have found is that the compositions containing low levels of oxidising agent which are also at low pH can be supplied in the form of a single phase product, that is a composition containing developers (i), couplers (A), (B) and/or (C) and inorganic peroxygen oxidising agent is storage stable at low pH.

Thus the composition of the invention can be supplied in the form of a single phase reusable composition when it is a low pH composition of the invention.

The colouring materials and oxidising agent undergo substantially no reaction in the composition, in particular when it is held at room temperature. When applied to the hair, however, the colouring components do react.

A further advantage of the low pH compositions of the invention when supplied in the form of a hair colouring kit containing the components of the composition in two or more separately packaged components is the stability of the components when mixed, if the composition is a low pH composition. Low pH compositions when mixed have the advantage that they remain stable for longer periods than do high pH compositions. Low pH compositions may therefore be mixed and remain usable for, for instance, up to half an hour or an hour. This allows additional time for the consumer to apply the mixed composition, so that, for instance, a first application may be made and subsequent “corrections” may also be made using the same pack of hair colouring material.

The developers, couplers, antioxidant and oxidising agent, and any other materials to be applied to the hair as components of the composition of the invention, may be provided in any suitable physical form. A preferred physical form is liquid. The liquid may be of low viscosity, for instance it may be water thin, or it may be of higher viscosity. The material may be suspended in a gel network. The gel may be solid or of low viscosity.

The materials for colouring the hair are often formulated so that when they are mixed to form the composition of the invention for application to the hair they form a product of cream like consistency, which is convenient for application to the hair. The final composition which is applied to the hair is often in the form of an emulsion.

Each individual material may be supplied in a form such that the composition containing it has a pH of above or below 7. For instance it may be from pH 1 to 11. In order to assist solubility of the various components, particularly developers and couplers, in a water-based carrier, the carrier may have a pH of above 6.1 or 6.5 or 7, for instance from pH 8 or 9 to pH 10 or 11. A pH as supplied of from 1 to 6 can assist in improving stability of the components.

The materials may be provided such that the pH of the final composition when mixed for application to the hair has a pH below 7 even though one of the components used to form it has a pH of above 7. Alcohols such as ethanol in amounts of from for instance 5 to 10 or 25% may be included to aid solubility of the developers and, particularly, the couplers in a water-based carrier.

The composition may also include an antioxidant, as described in our copending application number 9710754.4. Suitable antioxidants include sulphites such as sodium sulphite and chelants. Suitable amounts of antioxidant include from about 0.01 to 4 wt %, for instance about 0.1 to 1.5 wt %.

According to a second aspect of the invention there is provided a hair colouring kit comprising

(1) an individually packaged colouring component comprising

(i) one or more developers as defined above, and

(ii) one or more couplers selected from (A), (B) and (C) as defined above, and

(2) an individually packaged oxidising component,

wherein the individually packaged oxidising component contains inorganic peroxygen oxidising agent at such a molar level and the kit is provided such that when the colouring component and oxidising component are combined the combined composition contains inorganic peroxygen oxidising agent in an amount of from about 0.0001 moles to about 0.05 moles per 100 g of composition.

In this aspect of the invention it is important that the kit is provided so that the final composition produced will have the desired level of oxidising agent. This may be done for instance by supplying the kit such that the two individually packaged components need only be mixed to provide the correct final level. Alternatively, the kit may be supplied with instructions to the consumer to mix the individually packaged colouring component and individually packaged oxidising component in such a ratio that the desired final level of oxidising agent is achieved.

The individually packaged colouring component may itself be supplied in the form of separate components. For instance it may contain an individually packaged developer component, containing the one or more developer compounds (i) and an individually packaged coupler compound containing the coupler components (A), (B) and (C). Alternatively, the couplers may be supplied individually packaged, so that couplers (A) are supplied as a cyan component, couplers (B) are supplied as a yellow component and couplers (C) are supplied as a magenta component, together with instructions for mixing by the consumer to give a variety of different colours.

The components of the hair colouring kit of the second aspect of the invention may have any of the features described above for the composition of the invention.

In a third aspect of the invention we provide a method of colouring hair comprising providing

(i) one or more developers as defined above,

(ii) one or more couplers selected from (A), (B) and (C) as defined above and

(iii) an inorganic peroxygen oxidising agent

and applying (i), (ii), (iii) to the hair to be coloured, wherein the amount of inorganic peroxygen oxidising agent applied is from about 0.0001 moles to about 0.05 moles per 100 g of total composition applied.

Preferably the components are applied substantially simultaneously. In particular they are preferably mixed to form a single composition and then applied to the hair together. However, within the term “substantially simultaneously” we also include application of one or more components to the hair followed by subsequent application of the remaining components within a period of not more than 5 minutes.

Application of the components substantially simultaneously, in particular mixing and application together, is especially beneficial to the consumer because of the increased convenience over sequential application.

The conditions of the reaction are normally those conventionally applied for dyeing hair. The temperature is normally from 10 to 45° C., often 20 to 35° C. pH can be high (eg. above pH 6.1 or 6.5, often above pH 8 or 9 or even above 10) but is often low (eg. below pH 7 or 6).

In the specification, when leaving groups are discussed, as well as compounds which react only at one position or only with certain other compounds, we mean reaction under the conditions under which the colouring compounds will be applied to the hair.

In the method of the invention it is possible to use any of the components having any of the features discussed above for the composition of the invention.

In a fourth aspect of the invention we provide a hair colouring system which comprises

(i) one or more developers as defined above

(ii) one or more couplers selected from (a), (b) and (c) as defined above and

(iii) inorganic peroxygen oxidising agent in an amount of from about 0.0001 to about 0.05 moles per 100 g of composition which comprises no other oxidative colouring agents and the system is capable of providing a wide spectrum of colour shades without the use of additional colouring agents.

In the system of the fourth aspect of the invention, amounts and types of developer and coupler are selected so as to obtain the particular colour desired for any one application. For any different colour selection is made from the same set of developers and couplers to provide that colour. Preferably the system can provide at least one light brown shade, at least one red shade and at least one dark brown shade. More preferably it provides at least one blonde shade and at least one black shade.

Preferably the system comprises only one to four, preferably one to three or even only one or two, compounds of each of the types (i), (A), (B) and (C). Thus in this preferred embodiment of the system of the invention a wide range of colours is obtained from only a few, and even as few as four colouring components.

The system may also comprise instructions for selection of amounts and types of components (i), (ii) and (iii) to achieve a range of colours and degrees of permanence of coloration.

The system may be supplied to consumers, for instance those who wish to colour their own hair or to hairdressing salons, together with instructions to select particular amounts of each of the colouring components to obtain different colours or degrees of permanence of coloration. The system may also be used by manufacturers of hair colouring compositions. The manufacturer provides the four different types of colouring component and selects the amounts and types necessary for each individual colour which is intended to be marketed.

In the system of the invention any of the materials may be used which have been discussed above in the context of the composition of the invention.

The invention also includes a further aspect in which is provided a hair colouring composition comprising

(i) one or more developers selected from amino aromatic systems capable of being oxidised and thereafter undergoing a single electrophilic attack, and

(ii) one or more couplers selected from

(A) phenols and naphthols having an active leaving group in the para position relative to the hydroxyl group,

(B) 1,3-diketones containing the group

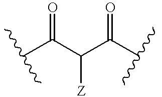

in which Z is an active leaving group, and

(C) compounds containing the group

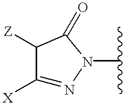

in which Z is an active leaving group, and X is an active leaving group or a non-leaving substituent,

such that in the presence of an oxidising agent the or each developer reacts with the or each coupler substantially only at the position having the active leaving group Z and, if X is an active leaving group, X,

and (iii) inorganic peroxygen oxidising agent,

wherein the composition has a pH of from 1 to 6.

In this aspect of the invention the composition may comprise conventional levels of oxidising agent provided it has pH below 6, which provides the advantages discussed above of low pH. In this aspect of the invention any of the features of the aspects of the invention discussed above may be used where relevant. The kit, method, and system of the first to fourth aspects of the invention may also be applied at low pH but with conventional levels of oxidising agent according to this further aspect of the invention.

In the compositions, methods, uses and systems of the invention any other conventional components of hair colouring compositions may be applied to the hair, for instance as described in our copending application number 9626713.3.

Any of the compositions can contain various optional ingredients as follows.

Oxidative Dye Precursors

Preferably the only oxidative dye materials in the composition are materials (i) and (ii) discussed above. However, the compositions may optionally contain minor amounts of other oxidative dye materials. These may include those described in our copending application PCT/US97/22719, filed Dec. 9, 1997.

In general terms, oxidative dye primary intermediates include those monomeric materials which, on oxidation, form oligomers or polymers having extended conjugated systems of electrons in their molecular structure. Because of the new electronic structure, the resultant oligomers and polymers exhibit a shift in their electronic spectra to the visible range and appear coloured. For example, oxidative primary intermediates capable of forming coloured polymers include materials such as aniline, which has a single functional group and which, on oxidation, forms a series of conjugated imines and quinoid dimers, trimers, etc. ranging in colour from green to black. Compounds such as p-phenylenediamine, which has two functional groups, are capable of oxidative polymerization to yield higher molecular weight coloured materials having extended conjugated electron systems. A representative list of primary intermediates and secondary couplers suitable for use herein is found in Sagarin, “Cosmetic Science and Technology”,” Interscience, Special Ed. Vol. 2 pages 308 to 310.

Non-oxidative and Other Dyes

The hair colouring compositions used in the present invention may, in addition to the essential oxidative hair colouring agents (i) and (ii) and optional oxidative dyes, optionally include non-oxidative and other dye materials. Optional non-oxidative and other dyes suitable for use in the hair colouring compositions and processes according to the present invention include both semi-permanent, temporary and other dyes. Non-oxidative dyes as defined herein include the so-called “direct action dyes”, metallic dyes, metal chelate dyes, fiber reactive dyes and other synthetic and natural dyes. Various types of non-oxidative dyes are detailed in: “Chemical and Physical Behaviour of Human Hair” 3rd Ed. by Clarence Robbins (pp250-259); “The Chemistry and Manufacture of Cosmetics”. Volume IV. 2nd Ed. Maison G. De Navarre at chapter 45 by G. S. Kass (pp841-920); “Cosmetics: Science and Technology”, 2nd Ed., Vol. II, Balsam Sagarin, Chapter 23 by F. E. Wall (pp 279-343); “The Science of Hair Care” edited by C. Zviak, Chapter 7 pp 235-261 and “Hair Dyes”, J. C. Johnson, Noyes Data Corp., Park Ridge, U.S.A. (1973), (pp 3-91 and 113-139).

Direct action dyes, which do not require an oxidative effect in order to develop the color, are also designated hair tints and have long been known in the art. They are usually applied to the hair in a base matrix which includes surf actant material. Direct action dyes include nitro dyes such as the derivatives of nitroamino benzene or nitroaminophenol; disperse dyes such as nitroaryl amines, aminoanthraquinones or azo dyes; anthraquinone dyes, naphthoquinone dyes; basic dyes such as Acridine Orange C.I. 46005.

Nitro dyes are added to dyeing compositions to enhance colour of colorant and to add suitable aesthetic colour to the dye mixture prior to application.

Further examples of direct action dyes include the Arianor dyes basic brown 17, C.I.(color index)—no. 12,251; basic red 76, C.I.—12,245; basic brown 16, C.I.—12,250; basic yellow 57, C.I.—12,719 and basic blue 99, C.I.—56,059 and further direct action dyes such as acid yellow 1, C.I.—10,316 (D&C yellow no.7); acid yellow 9, C.I.—13,015; basic violet C.I.—45,170; disperse yellow 3, C.I.—11,855; basic yellow 57, C.I.—12,719; disperse yellow 1, C.I.—10,345; basic violet 1, C.I.—42,535, basic violet 3, C.I.—42,555; greenish blue, C.I.—42090 (FD&C Blue no.1); yellowish red, C.I.—14700 (FD&C red no.4); yellow, C.I.19140 (FD&C yellow no5); yellowish orange, C.I.15985 (FD&C yellow no.6); bluish green, C.I.42053 (FD&C green no.3); yellowish red, C.I.16035 (FD&C red no.40); bluish green, C.I.61570 (D&C green no.3); orange, C.I.45370 (D&C orange no.5); red, C.I.15850 (D&C red no.6); bluish red, C.I.15850 (D&C red no.7); slight bluish red, C.I.45380 (D&C red no.22); bluish red, C.I.45410 (D&C red no.28); bluish red, C.I.73360 (D&C red no.30); reddish purple, C.I.17200 (D&C red no.33); dirty blue red, C.I.15880 (D&C red no.34); bright yellow red, C.I.12085 (D&C red no.36); bright orange, C.I.15510 (D&C orange no.4); greenish yellow, C.I.47005 (D&C yellow no.10); bluish green, C.I.59040 (D&C green no.8); bluish violet, C.I.60730 (Ext. D&C violet no.2); greenish yellow, C.I.10316 (Ext. D&C yellow no.7);

Fiber reactive dyes include the Procion (RTM), Drimarene (RTM), Cibacron (RTM), Levafix (RTM) and Remazol (RTM) dyes available from ICI, Sandoz, Ciba-Geigy, Bayer and Hoechst respectively.

Natural dyes and vegetable dyes as defined herein include henna (Lawsonia alba), camomile (Matricaria chamomila or Anthemis nobilis), indigo, logwood and walnut hull extract.

Temporary hair dyes, or hair coloring rinses, are generally comprised of dye molecules which are too large to diffuse into the hair shaft and which act on the exterior of the hair. They are usually applied via a leave-in procedure in which the dye solution is allowed to dry on the hair surface. As such these dyes are typically less resistant to the effects of washing and cleaning the hair with surface active agents and are washed off the hair with relative ease. Temporary hair dye can be used in the compositions of the invention and examples of preferred temporary hair dyes are illustrated below.

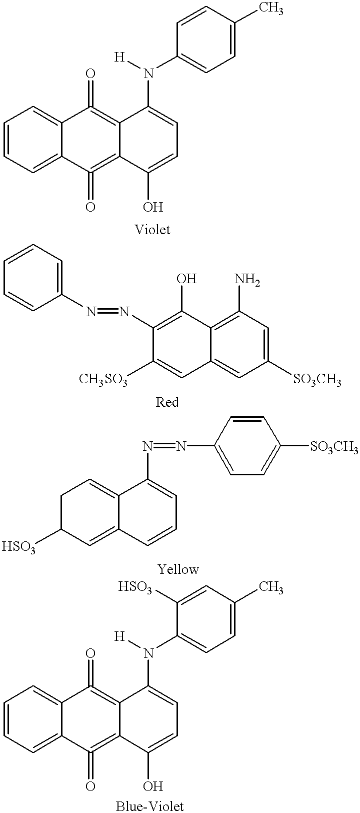

Semi-permanent hair dyes are dyes which are generally smaller in size and effect to temporary hair rinses but are generally larger than permanent (oxidative) dyes. Typically, semi-permanent dyes act in a similar manner to oxidative dyes in that they have the potential to diffuse into the hair shaft. However, semi-permanent dyes are generally smaller in size than the aforementioned conjugated oxidative dye molecules and as such are pre-disposed to gradual diffusion out of the hair again. Simple hair washing and cleaning action will encourage this process and in general semi-permanent dyes are largely washed out of the hair after about 5 to 8 washes. A semi-permanent dye system can be included in the compositions of the present invention. Suitable semi-permanent dyes for use in the compositions of the present invention are HC Blue 2, HC Yellow 4, HC Red 3, Disperse Violet 4, Disperse Black 9, HC Blue 7, HC Yellow 2, Disperse Blue 3, Disperse Violet 1 and mixtures thereof. Examples of semi-permanent dyes are illustrated below:

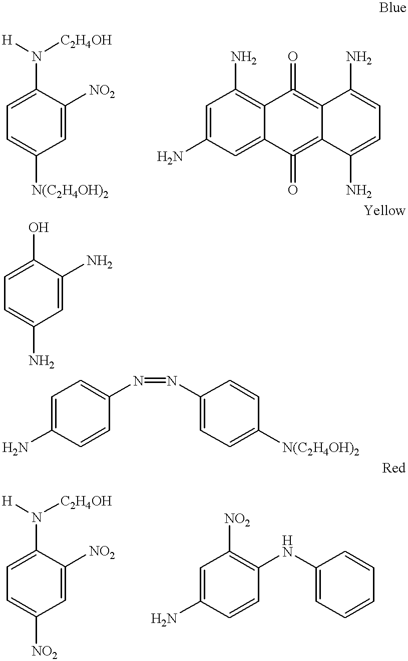

Typical semi-permanent dye systems incorporate mixtures of both large and small colour molecules. As the size of the hair is not uniform from root to tip the small molecules will diffuse both at the root and tip, but will not be retained within the tip, while the larger molecules will be generally only be able to diffuse into the ends of the hair. This combination of dye molecule size is used to help give consistent color results from the root to the tip of the hair both during the initial dyeing process and during subsequent washing.

Catalyst

The colouring compositions herein may optionally contain a catalyst for any inorganic peroxygen oxidising agents and the optional preformed peroxy acid oxidising agent(s).

Thickeners

The colouring compositions of the present invention may additionally include a thickener at a level of from about 0.05% to about 20%, preferably from about 0.1% to about 10%, more preferably from about 0.5% to about 5% by weight. Thickening agents suitable for use in the compositions herein are selected from oleic acid, cetyl alcohol, oleyl alcohol, sodium chloride, cetearyl alcohol, stearyl alcohol, synthetic thickeners such as Carbopol, Aculyn and Acrosyl and mixtures thereof. Preferred thickeners for use herein are Aculyn 22 (RTM,) steareth-20 methacrylate copolymer; Aculyn 44 (RTM) polyurethane resin and Acusol 830 (RTM), acrylate copolymers which are available from Rohm and Haas, Philadelphia, Pa., USA. Additional thickening agents suitable for use herein include sodium alginate or gum arabic, or cellulose derivatives, such as methyl cellulose or the sodium salt of carboxymethylcellulose or acrylic polymers.

Diluent

Water is the preferred diluent for the compositions according to the present invention. However, the compositions according to the present invention may include one or more solvents as additional diluent materials. Generally, solvents suitable for use in the coloring compositions of the present invention are selected to be miscible with water and innocuous to the skin. Solvents suitable for use as additional diluents herein include C₁-C₂₀ mono- or polyhydric alcohols and their ethers, glycerine, with monohydric and dihydric alcohols and their ethers preferred. In these compounds, alcoholic residues containing 2 to 10 carbon atoms are preferred. Thus, a preferred group includes ethanol, isopropanol, n-propanol, butanol, propylene glycol, ethylene glycol monoethyl ether, 1,2-hexanediol, butoxyethanol, benzyl alcohol, and mixtures thereof. Water is the preferred principal diluent in the compositions according to the present invention. Principal diluent, as defined herein, means, that the level of that diluent present is higher than the total level of any other diluents.

The diluent is present at a level preferably of from about 5% to about 99.98%, preferably from about 15% to about 99.5%, more preferably at least from about 30% to about 99%, and especially from about 50% to about 98% by weight of the compositions herein.

Enzyme

A further additional material useful in the hair coloring compositions according to the present invention is one or more enzymes.

Suitable enzymatic materials include the commercially available lipases, cutinases, amylases, neutral and alkaline proteases, esterases, cellulases, pectinases, lactases and peroxidases conventionally incorporated into detergent compositions. Suitable enzymes are discussed in U.S. Pat. Nos. 3,519,570 and 3,533,139.

Peroxidases are haemoproteins specific for peroxide, but using a wide range of substances as donors. Catalase which decomposes peroxide, is included here in view of the fact that it is generally similar in structure and properties and is able to bring about certain oxidations by H₂O₂. The decomposition of H₂O₂ can be regarded as the oxidation of one molecule by the other. It is widespread in aerobic cells and may have some more important function. The coenzyme peroxidases are not haemoproteins and one at least is a flavoprotein. Other flavoproteins such as xanthine oxidase will also use H₂O₂ among other acceptors, and the coenzyme peroxidases resemble these rather than the classical peroxidases in not being specific for H₂O₂. Suitable peroxidases for the compositions of the present invention include horseradish peroxidase, Japanese radish peroxidase, cow's milk peroxidase, rat liver peroxidase, linginase and haloperoxidase such as chloro- and bromo-peroxidase.

Enzymes are optionally incorporated at levels sufficient to provide up to about 50 mg by weight, more typically about 0.1 mg to about 10 mg of active enzyme per gram of the hair treatment composition of the invention. Stated otherwise the peroxidase enzyme may be incorporated into the compositions in accordance with the invention at a level of from about 0.0001% to about 5%, preferably from about 0.001% to about 1%, more preferably from about 0.01% to about 1% active enzyme by weight of the composition.

Commercially available protease enzymes include those sold under the trade names Alcalase, Savinase, Primase, Durazym, and Esperase by Novo Industries A/S (Denmark), those sold under the tradename Maxatase, Maxacal and Maxapem by Gist-Brocades, those sold by Genencor International, and those sold under the tradename Opticlean and Optimase by Solvay Enzymes. Protease enzyme may be incorporated into the compositions in accordance with the invention at a level of from 0.0001% to 4% active enzyme by weight of the composition.

Amylases include, for example, α-amylases obtained from a special strain of B.licheniformis, described in more detail in GB-1,269,839 (Novo). Preferred commercially available amylases include for example, those sold under the tradename Rapidase by Gist-Brocades, and those sold under the tradename Termamyl and BAN by Novo Industries A/S. Amylase enzyme may be incorporated into the composition in accordance with the invention at a level of from 0.0001% to 2% active enzyme by weight of the composition.

Lipolytic enzyme may be present at levels of active lipolytic enzyme of from 0.0001% to 2% by weight, preferably 0.001% to 1% by weight, most preferably from 0.001% to 0.5% by weight of the compositions.

The lipase may be fungal or bacterial in origin being obtained, for example, from a lipase producing strain of Humicola sp., Thermomyces sp. or Pseudomonas sp. including Pseudomonas pseudoalcaligenes or Pseudomonas fluorescens. Lipase from chemically or genetically modified mutants of these strains are also useful herein. A preferred lipase is derived from Pseudomonas pseudoalcaligenes, which is described in Granted European Patent, EP-B-0218272.

Another preferred lipase herein is obtained by cloning the gene from Humicola lanuginosa and expressing the gene in Aspergillus oryza, as host, as described in European Patent Application, EP-A-0258 068, which is commercially available from Novo Industri A/S, Bagsvaerd, Denmark, under the trade name Lipolase. This lipase is also described in U.S. Pat. No. 4,810,414, Huge-Jensen et al, issued Mar. 7, 1989.

Surfactant Materials

The compositions of the present invention can additionally contain a surfactant system. Suitable surfactants for inclusion in the compositions of the invention generally have a lipophilic chain length of from about 8 to about 22 carbon atoms and can be selected from anionic, cationic, nonionic, amphoteric, zwitterionic surfactants and mixtures thereof.

(i) Anionic Surfactants

Anionic surfactants suitable for inclusion in the compositions of the invention include alkyl sulphates, ethoxylated alkyl sulphates, alkyl glyceryl ether sulfonates, methyl acyl taurates, fatty acyl glycinates, N-acyl glutamates, acyl isethionates, alkyl sulfosuccinates, alkyl ethoxysulphosuccinates, alpha-sulfonated fatty acids, their salts and/or their esters, alkyl ethoxy carboxylates, alkyl phosphate esters, ethoxylated alkyl phosphate esters, alkyl sulphates, acyl sarcosinates, hydrotropes, such as alkyl xylene sulphonate and fatty acid/protein condensates, and mixtures thereof. Alkyl and/or acyl chain lengths for these surfactants are C₁₂-C₂₂, preferably C₁₂-C₁₄, more preferably C₁₂-C₁₄.

(ii) Nonionic Surfactants

The compositions of the invention can also comprise water-soluble nonionic surfactant(s). Surfactants of this class include C₁₂-C₁₄ fatty acid mono- and diethanolamides, sucrose polyester surfactants and polyhydroxy fatty acid amide surfactants having the general formula below.

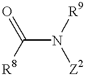

The preferred N-alkyl, N-alkoxy or N-aryloxy, polyhydroxy fatty acid amide surfactants according to the above formula are those in which R⁸ is C₅-C₃₁ hydrocarbyl, preferably C₆-C₁₉ hydrocarbyl, including straight-chain and branched chain alkyl and alkenyl, or mixtures thereof and R⁹is typically hydrogen, C₁-C₈ alkyl or hydroxyalkyl, preferably methyl, or a group of formula —R¹—O—R² wherein R¹ is C₂-C₈ hydrocarbyl including straight-chain, branched-chain and cyclic (including aryl), and is preferably C₂-C₄ alkylene, R² is C₁-C₈ straight-chain, branched-chain and cyclic hydrocarbyl including aryl and oxyhydrocarbyl, and is preferably C₁-C₈ alkyl, especially methyl, or phenyl. Z² is a polyhydroxyhydrocarbyl moiety having a linear hydrocarbyl chain with at least 2 hydroxyls (in the case of glyceraldehyde) or at least 3 hydroxyls (in the case of other reducing sugars) directly connected to the chain, or an alkoxylated derivative (preferably ethoxylated or propoxylated) thereof. Z² preferably will be derived from a reducing sugar in a reductive amination reaction, and most preferably Z² is a glycityl moiety. Suitable reducing sugars include glucose, fructose, maltose, lactose, galactose, mannose, and xylose, as well as glyceraldehyde. As raw materials, high dextrose corn syrup, high fructose corn syrup, and high maltose corn syrup can be utilised as well as the individual sugars listed above. These corn syrups may yield a mix of sugar components for Z². It should be understood that it is by no means intended to exclude other suitable raw materials. Z² preferably will be selected from the group consisting of —CH₂—(CHOH)ₙ—CH₂OH, —CH(CH₂OH)—(CHOH)ₙ₋₁—CH₂H, CH₂(CHOH)₂(CHOR′)CHOH)—CH₂OH, where n is an integer from 1 to 5, inclusive, and R′ is H or a cyclic mono- or polysaccharide, and alkoxylated derivatives thereof. As noted, most preferred are glycityls wherein n is 4, particularly —CH₂—(CHOH)₄—CH₂OH.

The most preferred polyhydroxy fatty acid amide has the formula R⁸(CO)N(CH₃)CH₂(CHOH)₄CH₂OH wherein R⁸ is a C6-C19 straight chain alkyl or alkenyl group. In compounds of the above formula, ^{R8}—CO—N< can be, for example, cocoamide, stearamide, oleamide, lauramide, myristamide, capricamide, palmiamide, tallowamide, etc.

Suitable oil-derived nonionic surfactants for use herein include water soluble vegetable and animal-derived emollients such as triglycerides with a polyethyleneglycol chain inserted; ethoxylated mono- and diglycerides, polyethoxylated lanolins and ethoxylated butter derivatives. One preferred class of oil-derived nonionic surfactants for use herein have the general formula below:

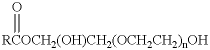

wherein n is from about 5 to about 200, preferably from about 20 to about 100, more preferably from about 30 to about 85, and wherein R comprises an aliphatic radical having on average from about 5 to 20 carbon atoms, preferably from about 7 to 18 carbon atoms.

Suitable ethoxylated oils and fats of this class include polyethyleneglycol derivatives of glyceryl cocoate, glyceryl caproate, glyceryl caprylate, glyceryl tallowate, glyceryl palmate, glyceryl stearate, glyceryl laurate, glyceryl oleate, glyceryl ricinoleate, and glyceryl fatty esters derived from triglycerides, such as palm oil, almond oil, and corn oil, preferably glyceryl tallowate and glyceryl cocoate.

Preferred for use herein are polyethyleneglycol based polyethoxylated C₉-C₁₅ fatty alcohol nonionic surfactants containing an average of from about 5 to about 50 ethyleneoxy moieties per mole of surfactant.

Suitable polyethylene glycol based polyethoxylated C₉-C₁₅ fatty alcohols suitable for use herein include C₉-C₁₁ Pareth-3, C₉-C₁₁, Pareth-4, C₉-C₁₁, Pareth-5, C₉-C₁₁ Pareth-6, C₉-C₁₁, Pareth-7, C₉-C₁₁, Pareth-8, C₁₁-C₁₅ Pareth-3, C₁₁-C₁₅ Pareth-4, C₁₁-C₁₅ Pareth-5, C₁₁-C₁₅ Pareth-6, C₁₁-C₁₅ Pareth-7, C₁₁-C₁₅ Pareth-8, C₁₁-C₁₅ Pareth-9, C₁₁-C₁₅ Pareth-10, C₁₁-C₁₅ Pareth-11, C₁₁-C₁₅ Pareth-12, C₁₁-C₁₅ Pareth-13 and C₁₁-C₁₅ Pareth-14. PEG 40 hydrogenated castor oil is commercially available under the tradename Cremophor (RTM) from BASF. PEG 7 glyceryl cocoate and PEG 20 glyceryl laurate are commercially available from Henkel under the tradenames Cetiol (RTM) HE and Lamacit (RTM) GML 20 respectively. C₉-C₁₁ Pareth-8 is commercially available from Shell Ltd under the tradename Dobanol (RTM) 91-8. Particularly preferred for use herein are polyethylene glycol ethers of ceteryl alcohol such as Ceteareth 25 which is available from BASF under the trade name Cremaphor A25.

Nonoxynol surfactants may also be used.

Also suitable for use herein are nonionic surfactants derived from composite vegetable fats extracted from the fruit of the Shea Tree (Butyrospermum Karkii Kotschy) and derivatives thereof. Similarly, ethoxylated derivatives of Mango, Cocoa and Illipe butter may be used in compositions according to the invention. Although these are classified as ethoxylated nonionic surfactants it is understood that a certain proportion may remain as non-ethoxylated vegetable oil or fat.

Other suitable oil-derived nonionic surfactants include ethoxylated derivatives of almond oil, peanut oil, rice bran oil, wheat germ oil, linseed oil, jojoba oil, oil of apricot pits, walnuts, palm nuts, pistachio nuts, sesame seeds, rapeseed, cade oil, corn oil, peach pit oil, poppyseed oil, pine oil, castor oil, soybean oil, avocado oil, safflower oil, coconut oil, hazelnut oil, olive oil, grapeseed oil, and sunflower seed oil.

(iii) Amphoteric Surfactants

Amphoteric surfactants suitable for use in the compositions of the invention include:

(a) imidazolinium surfactants of the formula (1)

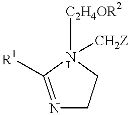

wherein R¹ is C₇-C₂₂ alkyl or alkenyl, R² is hydrogen or CH₂Z, each Z is independently CO₂M or CH₂CO₂M, and M is H, alkali metal, alkaline earth metal, ammonium or alkanolammonium; and/or ammonium derivatives of the formula (2)

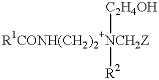

wherein R¹, R² and Z are as defined above;

(b) aminoalkanoates of the formula (3)

R¹NH (CH₂)ₙCO₂M

iminodialkanoates of the formula (4)

R¹N [(CH₂)ₘCO₂M]₂

and iminopolyalkanoates of the formula (5)

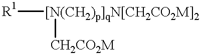

wherein n, m, p, and q are numbers from 1 to 4, and R¹ and M are independently selected from the groups specified above; and

(c) mixtures thereof.

Suitable amphoteric surfactants of type (a) are marketed under the trade name Miranol and Empigen and are understood to comprise a complex mixture of species. In practice, a complex mixture of cyclic and non-cyclic species is likely to exist and both definitions are given here for sake of completeness. Preferred for use herein, however, are the non-cyclic species.

Examples of suitable amphoteric surfactants of type (a) include compounds of formula (1) and/or (2) in which R₁ is C₈H₁₇ (especially iso-capryl), C₉H₁₉ and C₁₁H₂₃ alkyl. Especially preferred are the compounds in which R¹ is C₉H₁₉, Z is CO₂M and R² is H; the compounds in which R¹ is C₁₁H₂₃, Z is CO₂M and R² is CH₂CO₂M; and the compounds in which R¹ is C₁₁H₂₃, Z is CO₂M and R² is H.

In CTFA nomenclature, materials suitable for use in the present invention include cocoamphocarboxypropionate, cocoamphocarboxy propionic acid, and especially cocoamphoacetate and cocoamphodiacetate (otherwise referred to as cocoamphocarboxyglycinate). Specific commercial products include those sold under the trade names of Ampholak 7TX (sodium carboxy methyl tallow polypropyl amine), Empigen CDL60 and CDR 60 (Albright & Wilson), Miranol H2M Conc. Miranol C2M Conc. N.P., Miranol C2M Conc. O.P., Miranol C2M SF, Miranol CM Special (Rhône-Poulenc); Alkateric 2CIB (Alkaril Chemicals); Amphoterge W-2 (Lonza, Inc.); Monateric CDX-38, Monateric CSH-32 (Mona Industries); Rewoteric AM-2C (Rewo Chemical Group); and Schercotic MS-2 (Scher Chemicals). Further examples of amphoteric surfactants suitable for use herein include Octoxynol-l (RTM), polyoxethylene (1) octylphenyl ether; Nonoxynol-4 (RTM), polyoxyethylene (4) nonylphenyl ether and Nonoxynol-9, polyoxyethylene (9) nonylphenyl ether.

It will be understood that a number of commercially-available amphoteric surfactants of this type are manufactured and sold in the form of electroneutral complexes with, for example, hydroxide counterions or with anionic sulfate or sulfonate surfactants, especially those of the sulfated C₈-C₁₈ alcohol, C₈-C₁₈ ethoxylated alcohol or C₈-C₁₈ acyl glyceride types. Note also that the concentrations and weight ratios of the amphoteric surfactants are based herein on the uncomplexed forms of the surfactants, any anionic surfactant counterions being considered as part of the overall anionic surf actant component content.

Examples of preferred amphoteric surfactants of type (b) include N-alkyl polytrimethylene poly-, carboxymethylamines sold under the trade names Ampholak X07 and Ampholak 7CX by Berol Nobel and also salts, especially the triethanolammonium salts and salts of N-lauryl-beta-amino propionic acid and N-lauryl-imino-dipropionic acid. Such materials are sold under the trade name Deriphat by Henkel and Mirataine by Rhône-Poulenc.

(iv) Zwitterionic Surfactants

Water-soluble auxiliary zwitterionic surfactants suitable for inclusion in the compositions of the present invention include alkyl betaines of the formula R⁵R⁶R⁷N⁺(CH₂)ₙCO₂M and amido betaines of the formula (6) below:

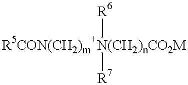

wherein R⁵ is C₁₁-C₂₂ alkyl or alkenyl, R⁶ and R⁷ are independently C₁-C₃ alkyl, M is H, alkali metal, alkaline earth metal, ammonium or alkanolammonium, and n, m are each numbers from 1 to 4. Preferred betaines include cocoamidopropyldimethylcarboxymethyl betaine, laurylamidopropyldimethylcarboxymethyl betaine and Tego betaine (RTM).

Water-soluble auxiliary sultaine surfactants suitable for inclusion in the compositions of the present invention include alkyl sultaines of the formula (7) below:

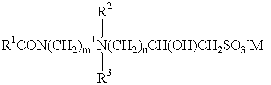

wherein R¹ is C₇ to C₂₂ alkyl or alkenyl, R² and R³ are independently C₁ to C₃ alkyl, M is H, alkali metal, alkaline earth metal, ammonium or alkanolammonium and m and n are numbers from 1 to 4. Preferred for use herein is coco amido propylhydroxy sultaine.

Water-soluble auxiliary amine oxide surfactants suitable for inclusion in the compositions of the present invention include alkyl amine oxide R⁵R⁶R⁷NO and amido amine oxides of the formula (8) below:

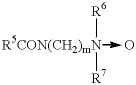

wherein R⁵ is C₁ to C₂₂ alkyl or alkenyl, R and R⁷ are independently C₁ to C₃ alkyl, M is H, alkali metal, alkaline earth metal, ammonium or alkanolammonium and m is a number from 1 to 4. Preferred amine oxides include cocoamidopropylamine oxide, lauryl dimethyl amine oxide and myristyl dimethyl amine oxide.

Additional Optional Materials

A number of additional optional materials can be added to the coloring compositions herein described, each at a level of from about 0.001% to about 5%, preferably from about 0.01% to about 3%, more preferably from about 0.05% to about 2% by weight of composition. Such materials include proteins and polypeptides and derivatives thereof; water-soluble or solubilizable preservatives; natural preservatives such as benzyl alcohol, potassium sorbate and bisabalol, benzoic acid, sodium benzoate and 2-phenoxyethanol; dye removers such as oxalic acid, sulphated castor oil, salicylic acid and sodium thiosulphate; H₂O₂ stabilisers; moisturising agents such as hyaluronic acid, chitin , and starch-grafted sodium polyacrylates such as Sanwet (RTM) IM-1000, IM-1500 and IM-2500 available from Celanese Superabsorbent Materials, Portsmith, Va., USA and described in U.S. Pat. No. 4,076,663 as well as methyl cellulose, starch, higher fatty alcohols, paraffin oils, fatty acids and the like; solvents; anti-bacterial agents such as Oxeco (phenoxy isopropanol); low temperature phase modifiers such as ammonium ion sources (e.g. NH₄ Cl); viscosity control agents such as magnesium sulfate and other electrolytes; quaternary amine compounds such as distearyl-, dilauryl-, di-hydrogenated beef tallow-, dimethyl ammonium chloride, dicetyldiethyl ammoniumethylsulphate, ditallowdimethyl ammonium methylsulphate, disoya dimethyl ammonium chloride and dicoco dimethyl ammonium chloride; hair conditioning agents such as silicones, higher alcohols, cationic polymers and the like; enzyme stabilisers such as water soluble sources of calcium or borate species; colouring agents; TiO₂ and TiO₂-coated mica; perfumes and perfume solubilizers; and zeolites such as Valfour BV400 and derivatives thereof and Ca²⁺/Mg²⁺ sequestrants such as polycarboxylates, amino polycarboxylates, polyphosphonates, amino polyphosphonates etc. and water softening agents such as sodium citrate. Other optional materials include anti-dandruff actives such as ZPT, and perfumes.

### EXAMPLES

The invention will now be illustrated by the following examples. In these examples, various standard tests are used, as follows.

I Assessment of Initial Colour and Colour Change (Measurement of ΔE)

The equipment used to measure both the initial colour and colour change of substrates (hair/skin) dyed with the low pH colouring compositions of the present invention is a Hunter Colourquest spectrophotometer. The value used to express the degree of colour change on any particular substrate is Delta E (ΔE). Delta E, as defined herein, is represented by a factual sum of L, a, and b values such that:

ΔE=(ΔL²+Δa²+Δb²)¹

and L is measure of lightness and darkness (colour intensity), wherein L=100 is equivalent to white, and L=0 is equivalent to black. Further, “a” is a measure of the red and green quotients (colour hues) such that positive equates to red and negative to green and “b” is a measure of the yellow and blue quotients (colour hues) such that positive equates to yellow and negative equates to blue.

Hunter Colourquest measurements can be carried out on the Hunter Labscan Colourimeter which is a full scanning spectrocolorimeter with a wavelength of from 400-700 nanometers which records the colour of test hair switches (tresses) in terms of “L”, “a” and “b” values. The machine is set to: mode—0/45; port size—1 inch; view size—1 inch; light—D65; field of view—10°; UV lamp/filter—none. The hair is placed in a sample holder designed to hold the hair in uniform orientation during measurement. Equivalent calorimeters can be used, but it must be ensured that the hair does not move during measurement. The hair must be spread to cover the 1 inch port during colour measurement. Dots are placed on the switch holder to guide the positioning of the holder at the port. The dots are lined up with a mark on the port and readings are taken at each spot.

Eight measurements are run per switch, 4 on each side, and three switches are run per treatment.

II Standard Hair Switch

The compositions according to the present invention can be used to colour hair of all colours, types and condition. For the purposes of illustration various switches can be tested. Two test hair switches can be measured in terms of their approximate L, a, b values.

| | L | a | b |
|---|---|---|---|
| Light brown | about 60 | about 9 | about 32 |
| (permed and bleached) | | | |
| 40% grey dark brown | 35-37 | 4.5-5.5 | 11.5-12.7 |

Yak hair (virgin or permed and/or bleached) can also be used. It has values of: L=about 82 to 83, a=about −0.5 to −0.7, b=about 11 to 12.

III Hair Switch Colouring Method

To colour hair, a 4 gramme switch of about 8 inch long hair (or a 2 gramme switch of 4 inch long hair) is hung over a suitable container. The test colouring product is then prepared (ie, where applicable the separate bottle components are mixed together) and about 2 grammes of product per gramme hair is applied directly to the test hair switch. The colourant is massaged through the hair switch for up to about 1 minute and then left on the hair switch for up to about 30 minutes. After rinsing with running water for about 1 or 2 minutes the coloured hair switch is then cleansed (according to the shampoo protocol IV below) and dried. Drying can be effected either naturally (without heat assistance) or using a drier. The colour development (initial colour) of the coloured, cleansed, dried test hair switch can then be assessed using the Hunter Colourquest spectrophotometer.

For the delivery of a red shade (hue) to prepermed, prebleached light brown hair (having L, a, b values of approximately 60, 9 and 32) the preferred initial shade of the coloured hair will have a hue value (arc tangent of (b/a)) in the range of from about 25 to about 70, more preferably from about 30 to about 65, most preferably from about 35 to about 60 and wherein the initial colour intensity (L) is greater than about 10 and less than about 70, preferably greater than about 15 and less than about 65 more preferably greater than about 20 and less than about 60.

For the delivery of a brown or black shade (hue) to prepermed, prebleached light brown hair (having L, a, b values of approximately 60, 9 and 32) the preferred initial shade of the coloured hair will have a hue value (arc tangent of (b/a)) of less than about 25, preferably less than about 20 and the initial colour intensity (L) will be greater than about 1 and less than about 50, preferably greater than about 5 and less than about 45.

For the delivery of a light brown shade (hue) to prepermed, prebleached light brown hair (having L, a, b values of approximately 60, 9 and 32) the preferred initial shade of the coloured hair will have a hue value (arc tangent of (b/a)) in the range of from about 70 up to about 110 and wherein the initial colour intensity (L) will be greater than about 20 and less than about 95, preferably greater than about 25 and less than about 90.

A significant colour change, as delivered via the colouring compositions according to the present invention preferably means a colour change on permed and bleached hair in terms of Delta E which is greater than about 5 or 8, preferably greater than about 10, more preferably greater than about 12, most preferably greater than about 15 and especially greater than about 20.

IV Hair Switch Cleansing Method

Switches of coloured hair are subjected to a repeated cleansing cycle wherein the following process is repeated.

A 4 gramme, 8 inch test switch (or a 2 gramme, 4 inch test switch) of coloured hair is clamped over a suitable container and rinsed thoroughly for about 10 seconds using warm water (at about 100° F. at about 1.5 gallons/minute pressure). Shampoo (about 0.1 ml non-conditioning shampoo per gramme hair) can then be applied directly to the wet test switch using a syringe. After lathering the hair for about 30 seconds the hair is rinsed in running water for about 30 seconds. The shampoo and lathering process is then repeated with a final 60 second rinse. Excess water can be removed (squeezed) from the test switch using the fingers. The test switch is then dried either naturally, or using a pre-heated dryer box at about 140° F. (for about 30 minutes). The coloured, cleansed, dried test hair switch can then be colour assessed (Delta E fade).

During any single test cycle each different switch to be assessed should be tested in water equivalent temperature, pressure level and hardness level.

Delta E fade results for prepermed, prebleached light brown hair (having L, a, b values approximately 60, 9 and 32) which has been coloured a red shade (of hue value in the range of from about 25 to about 70) are preferably less than about 5.0, preferably less than about 4.5, more preferably less than about 4.0, particularly preferably less than about 3.0 or 2.0 and wherein the change in hair colour, % delta E, after up to 20 washes, is less than about 20%, and preferably less than about 15%, more preferably less than about 10%.

Delta E fade results for prepermed, prebleached light brown hair (having L, a, b values of approximately 60, 9 and 32) which has been coloured a brown or black shade (of hue value of less than about 25) are preferably less than about 2.3, preferably less than about 2.0, more preferably less than about 1.7 and wherein the change in hair colour, % delta E, after up to 20 washes, is less than about 5%, preferably less than about 4.5%, more preferably less than about 4%, most preferably less than about 3.5%.

Delta E fade results for prepermed, prebleached light brown hair (having L, a, b values of approximately 60, 9 and 32) which has been coloured a light brown shade (of hue in the range of from about 70 to about 110) are preferably less than about 2.6, preferably less than about 2.3 and wherein the change in hair colour, % delta E, after up to 20 washes, is less than about 15%, preferably less than about 12%, more preferably less than about 10%, most preferably less than about 8%.

In preferred embodiments herein, the change in the colour of the dyed hair over time (Delta E fade) is less than about 15%, preferably less than about 12%, more preferably less than about 10% and most preferably less than about 8%.

V Perming Protocol

The following method is used to perm hair which is usually subsequently to be bleached.

A 4 gram switch of about 8 inch long hair is hung over a suitable container. Perming solution supplied under the trade name “Zotos” is applied to the hair so as to saturate it totally. The switches are then resaturated. The switches are then laid on a plastic tray for 20 minutes and subsequently rinsed for 1½ to 2 minutes with tap water at 37° C. The switches are squeezed dry and towelled dry. The switches are then hung over the container again and commercially available “Zotos” neutraliser is applied so as to saturate them. They are then laid in the plastic tray for 5 minutes and subsequently rinsed for 1½ to 2 minutes in tap water at 37° C. The switches are then shampooed twice and left to dry.

VI Bleaching Protocol

The prepermed switches are dried for 20 minutes and hung over the edge of the container. A maximum of 9 or 10 switches at once are treated. The commercially available bleach from Clairol, “Born Blonde (with chamomile)” is mixed according to the instructions and 10 grams of the material is applied to each switch and massaged in thoroughly. Each switch is wrapped loosely in clingfilm and left for 30 minutes. It is subsequently rinsed for 2 minutes in tap water at 37° C. It is then shampooed once.

### Example 1

In this example a low oxidising agent, low pH composition 1 of the invention is demonstrated. Additionally it is compared with a high pH commercially available product for skin staining properties.

The following components are used in formulation 1 as colouring components:

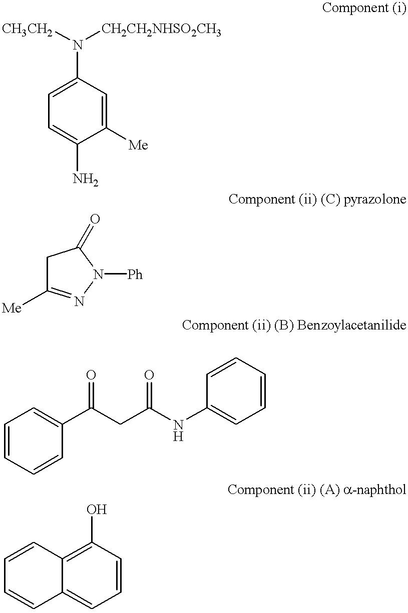

Formulation 1 of the invention has the following composition:

| | % by weight |
|---|---|
| Ceteareth 25 | 1.57 |
| Cetyl alcohol | 2.36 |
| Stearyl alcohol | 2.36 |
| (i) | 2.1 |
| Pyrazolone (ii) (C) | 0.22 |
| α-naphthol (ii) (A) | 0.012 |
| Benzoylacetanilide (ii) (B) | 0.61 |
| Ethanol | 7.7 |
| NH₄OH | 0 |
| Hydrogen Peroxide | 1.1 |
| Sodium Sulphite | 0.4 |
| EDTA | 0.4 |
| Water | up to 100 |
| The pH of formulation 1 is 3.8. | |

This was compared with a commercially available high oxidising agent, high pH composition, Recital “Santiago”, available from L'Oreal.

Before dyeing, the values of L, a and b of the human skin to be tested were measured. 0.2 g/cm³ were applied to human skin and left for 30 minutes. The composition was then rinsed off and the values of L, a and b were measured so as to calculate Delta E initial, which represents the difference in colour between the skin before dyeing and the skin after dyeing.

Results are given in Table 1 below.

| | L | a | b | ΔE initial | |
|---|---|---|---|---|---|
| Formulation 1 | | | | | |
| skin initial | 64.5 | 10.6 | 11.9 | | (No Visible |
| skin after dyeing | 63.21 | 10.5 | 12.8 | 1.6 | Staining) |
| L'Oreal Santiago | | | | | |
| skin initial | 65.8 | 11.3 | 13.0 | | (Considerable |
| skin after dyeing | 55.4 | 16.6 | 15.2 | 12.00 | Staining) |

It can be seen from Table 1 that the value of Delta E is significantly greater using the commercially available formulation than using the formulation 1 of the invention. The commercial formulation showed considerable skin staining, whereas formulation 1 of the invention showed essentially no visible staining even after application for 30 minutes.

### Example 2

The following formulation was tested:

| | | |
|---|---|---|
| Stearyl Alcohol | | 2.7% |
| Cetyl Alcohol | | 2.7% |
| Ceteareth-25 | | 1.8% |
| Ammonium Hydroxide | | 0% |
| N-Ethyl(-N-methanesulphonamide | | 3% |
| ethyl)-2-methyl- | | |
| p-phenylenediamine | | |
| Phenylmethylpyrazolinone | | 0.17% |
| Acetoacetanilide | | 1.3% |
| 1-Naphthol | | 0.004% |
| Sodium Sulphite | | 0.2% |
| EDTA | | 0.2% |
| Water | up to | 100% |
| pH | | 4.5 |

The formulation was applied to a permed and bleached light brown switch for 30 minutes.

Results were as follows:

L=27.4, a=14.7, b=11.5, ΔE (uptake)=39.4. The colour produced was a dark auburn.

### Example 3

The following formulation was tested:

| | | |
|---|---|---|
| Stearyl Alcohol | | 1.62% |
| Cetyl Alcohol | | 1.62% |
| Ceteareth-25 | | 1.11% |
| Ammonium Hydroxide | | 1.8% |
| 2,6-Dichloro-p-aminophenyl-HCl | | 1.0% |
| 3-Methyl-pyrazolinone | | 0.8% |
| N,N-Dimethyl acetoacetamide | | 2% |
| n-Propyl Gallate | | 0.8% |
| Asorbic Acid | | 0.4% |
| EDTA | | 0.2% |
| Hydrogen Peroxide | | 3% |
| Citric Acid | | 0.24% |
| Water | up to | 100% |
| pH | | 5.2 |

It was applied to permed yak hair and permed and bleached light brown hair. Results were as follows:

| Switch | | L | a | b | ΔE |
|---|---|---|---|---|---|
| Yak (permed) | before dyeing | 82.8 | −0.6 | 11.7 | 33.1 |
| Yak (permed) | after dyeing | 50.6 | 6.1 | 15.6 | 33.1 |
| | | (silvery brown colour) | | | |
| Light Brown | before dyeing | 60.1 | 9.7 | 32.1 | |
| (permed and | | | | | |
| bleached) | | | | | |
| Light Brown | after dyeing | 43.9 | 11.1 | 24.4 | 18.1 |
| (permed and | | | | | |
| bleached) | | | | | |

dark blonde colour

1. A hair colouring composition comprising
(i) one or more developers selected from aminoaromatic systems capable of being oxidised and thereafter undergoing a single electrophilic attack, and
(ii) one or more couplers selected from
(A) phenols and naphthols having an active leaving group in the para position relative to the hydroxy group,
(B) 1,3-diketones containing the group 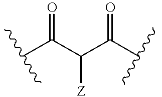
in which Z is an active leaving group, and
(C) compounds containing the group 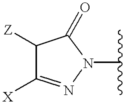
in which Z is an active leaving group, and X is an active leaving group or a non-leaving substituent,
such that in the presence of an oxidising agent the or each developer reacts with the or each coupler substantially only at the position having the active leaving group Z and, if X is an active leaving group, X,
and (iii) inorganic peroxygen oxidising agent in an amount of from about 0.0001 to about 0.05 moles per 100 g of composition:

2. A hair colouring composition according to claim 1 in which the one or more developers (i) are selected from N,N-disubstituted p-phenylene diamines.

3. A composition according to claim 1 in which the one or more developers (i) are selected from compounds of the formula 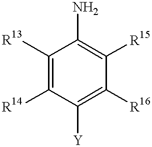
and compounds of the formula 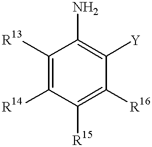
in which Y is selected from —NO₂, —CO₂H, —CO₂R, —COR and OH and R¹³, R¹⁴, R¹⁵ and R¹⁶ are independently selected from H, methyl, ethyl, n-propyl, i-propyl, F, Cl, OH, —CO₂H, —CO₂R and —COR.

4. A composition according to any preceding claim in which the one or more couplers (A) are selected from compounds having the formula 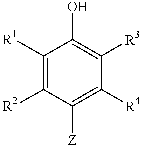
in which R¹, R², R³ and R⁴ are independently selected from OH, H, methyl, ethyl, n-propyl, i-propyl, t-butyl, —CO₂H, and —COR.

5. A composition according to any preceding claim in which coupler (B) is selected from compounds of the formula 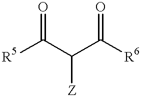
in which R⁵ is methyl, ethyl, n-propyl, i-propyl, t-butyl or phenyl and R⁶ is NR₂ in which the groups R are independently methyl, ethyl, n-propyl, i-propyl, t-butyl or phenyl or R⁵ is C₁₋₄ alkyl and R⁶ is C₁₋₄ alkyl or C₁₋₄ alkoxy.

6. A composition according to any preceding claim in which coupler (C) is selected from compounds having the formula 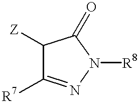
in which R⁷ and R⁸ are independently methyl, ethyl, n-propyl, i-propyl, t-butyl, substituted phenyl or unsubstituted phenyl.

7. A composition according to any preceding claim which includes at least one coupler of type (B) and/or at least one coupler of type (C).

8. A composition according to any preceding claim which includes at least one coupler of type (B).

9. A composition according to any preceding claim which includes couplers of at least two of the types (A), (B) and (C).

10. A composition according to any preceding claim which includes at least one coupler of each of the types (A), (B) and (C).

11. A composition according to any preceding claim which includes developer (i) in an amount of from 0.01 to 7 wt %, coupler (A) in an amount of from 0.001 to 1 wt %, coupler (B) in an amount of from 0.005 to 4 wt % and/or coupler (C) in an amount of from 0.01 to 4 wt %.

12. A composition according to any preceding claim which comprises only one developer compound (i).

13. A composition according to any preceding which includes not more than two couplers of each of the types (A), (B) and (C).

14. A composition according to any preceding claim which contains not more than 0.1 wt % oxidative colouring agents which are not of the types (i), (A), (B) and (C).

15. A composition according to any preceding claim which contains not more than 0.1 wt % oxidative colouring agents which are capable of undergoing reaction more than once under the conditions of hair colouring.

16. A composition according to any preceding claim in which X in coupler (C) is a non-leaving substituent.

17. A composition according to any preceding claim which has a pH of from about 1.5 to about 5.8.

18. A composition according to any preceding claim containing below 0.3 wt % ammonia.

19. A composition according to any of claims 1 to 16 which has a pH of at least 6.1, preferably at least 6.5.

20. A composition according to any of claims 1 to 16 which contains ammonia or ammonium hydroxide in an amount of at least 0.01 wt %.

21. A composition according to any preceding claim in which the oxidising agent is hydrogen peroxide.

22. A composition according to any preceding claim which comprises not more than 0.1 wt % of any oxidative colouring agent which can react with itself under the conditions of hair colouring.

23. A composition according to any preceding claim in which the developer molecules (i) react substantially only with the coupler molecules (A), (B) and/or (C), and not with other developer molecules, under the conditions of hair colouring.

24. A hair colouring kit comprising
(1) an individually packaged colouring component comprising
(i) one or more developers as defined in claim 1, and
(ii) one or more couplers as defined in claim 1, and
(2) an individually packaged oxidising component,
wherein the individually packaged oxidising component contains inorganic peroxygen oxidising agent at such a molar level and the kit is provided such that when the colouring component and oxidising component are combined the combined composition contains inorganic peroxygen oxidising agent in an amount of from about 0.0001 moles to about 0.05 moles per 100 g of composition.

25. A method of colouring hair comprising providing
(i) one or more developers as defined in claim 1, and
(ii) one or more couplers as defined in claim 1, and
(iii) an inorganic peroxygen oxidising agent and applying (i), (ii), (iii) to the hair to be coloured, wherein the amount of inorganic peroxygen oxidising agent applied is from about 0.0001 moles to about 0.05 moles per 100 g of total composition applied.

26. A method according to claim 25 in which the hair has previously been permed.

27. A system for colouring hair comprising
(i) one or more developers as defined in claim 1, and
(ii) one or more couplers as defined in claim 1, and
(iii) inorganic peroxygen oxidising agent in an amount of from about 0.0001 to about 0.05 moles per 100 g of composition, which comprises no other oxidative colouring agents and the system is capable of providing a wide spectrum of colour shades without the use of additional colouring agents.

28. A hair colouring composition comprising
(i) one or more developers selected from aminoaromatic systems capable of being oxidised and thereafter undergoing a single electrophilic attack, and
(ii) one or more couplers selected from
(A) phenols and naphthols having an active leaving group in the para position relative to the hydroxy group,
(B) 1,3-diketones containing the group 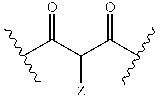
in which Z is an active leaving group, and
(C) compounds containing the group 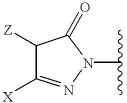
in which Z is an active leaving group, and X is an active leaving group or a non-leaving substituent,
such that in the presence of an oxidising agent the or each developer reacts with the or each coupler substantially only at the position having the active leaving group Z and, if X is an active leaving group, X,
and (iii) inorganic peroxygen oxidising agent
wherein the composition has a pH of not more than 6.

29. A hair colouring kit comprising
(1) an individually packaged colouring component comprising
(i) one or more developers as defined in claim 28, and
(ii) one or more couplers as defined in claim 28, and
(2) an individually packaged oxidising component,
wherein the kit is such that when the components (1) and (2) are mixed the mixture has a pH of not more than 6.

30. A method of colouring hair comprising providing
(i) one or more developers as defined in claim 28, and
(ii) one or more couplers as defined in claim 28, and
(iii) an inorganic peroxygen oxidising agent and applying (i), (ii), (iii) to the hair to be coloured, wherein the mixture applied to the hair has a pH of not more than 6.

31. A kit, method or system according to any of claims 24 to 27 additionally having any of the features set out in claims 2 to 20.

32. A composition, kit or method according to any of claims 28 to 30 additionally having any of the features set out in claims 2 to 18 and 21 to 23.

33. A composition, kit, method or system according to any preceding claim in which the developer (i) is selected from compounds which contain a single primary amine group and react only at the primary amine group under the conditions of hair colouring.
